(19) United States
(12) Patent Application Publication
(10) Pub. No.: US 2008/0233056 A1
(43) Pub. Date: Sep. 25, 2008

(54) ORGANOLEPTIC COMPOUNDS WITH ENHANCED PROPERTIES

(75) Inventor: Volker Berl, New York, NY (US)

Correspondence Address: MORGAN, LEWIS & BOCKIUS LLP (SF), One Market, Spear Street Tower, Suite 2800, San Francisco, CA, 94105, US

(73) Assignee: ZYMES, LLC, Hasbrouck Heights, NJ (US)

(21) Appl. No.: 12/051,797

(22) Filed: Mar. 19, 2008

Related U.S. Application Data
(60) Provisional application No. 60/895,681, filed on Mar. 19, 2007.

(51) Int. Cl. A61K 8/06; C11D 3/50; A61K 8/63; A61K 8/49; A61K 47/08; A61Q 13/00; A23L 1/22; A23L 2/56

(52) U.S. Cl. 424/49; 512/4; 514/772; 510/101; 424/484; 426/536

(57) ABSTRACT

The present invention provides a method of enhancing an organoleptic property of a composition by solubilizing the organoleptic additive in the composition using one or more solubilizing agent. An exemplary solubilizing agent has the general formula:

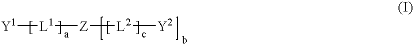

wherein a, b and c are integers independently selected from 0 and 1. Z is a member selected from a sterol, a tocopherol, a ubiquinol and derivatives or homologues thereof. Y¹ and Y² are hydrophilic moieties, which are members independently selected from polyethers, polyalcohols and derivatives thereof, and L¹ and L² are linker moieties independently selected from substituted or unsubstituted alkyl and substituted or unsubstituted heteroalkyl.

### CROSS REFERENCES TO RELATED APPLICATIONS

This application claims priority under 35 U.S.C. §119(e) to U.S. Provisional Patent Application No. 60/895,681, filed on Mar. 19, 2007, the disclosure of which is incorporated by reference herein in its entirety for all purposes.

### BACKGROUND OF THE INVENTION

The present invention relates to a process for rendering organoleptic compounds water soluble, thereby enhancing the organoleptic properties of these compounds.

A frequent problem associated with the application of flavor and fragrance systems is the lack of solubility of one or more organoleptic additive in the carrier. Furthermore, preparations of organoleptic additives frequently degrade and lose flavor/odor by volatilization or chemical decomposition. The loss of flavor usually results in flavor profile distortion or even in complete loss of flavor. Therefore, food scientists and application specialists are continuously searching for methods to enhance the solubility of organoleptic additives and to protect flavoring agents against volatilization and decomposition.

Another category of flavor application problems results from differences in the interaction between the flavoring agent and the product base. These differences in the flavor-matrix interactions result also in flavor distortion due to the different rates of flavor release during consumption of the product. Typical examples of this type of flavor application problems are the change of flavor character and strength in chewing gum during mastication and the flavor imbalance observed when applying standard flavoring agents to low fat products.

One of the preferred methods to control flavor retention and release is encapsulation. A considerable amount of effort has been devoted for many years to provide solid particulate flavoring materials in which a flavor is contained in the particulate matrix. Various attempts have been made to fix the flavors in many different types of organic matrices to provide stable free-flowing powders of particles which contain the flavor for flavor release when incorporated in foods. Another approach consists of dissolving a an organoleptic additive in a water-miscible organic solvent, such as ethanol or propylene glycol. However, when such a solution comes into contact with blood or gastrointestinal fluids, the organoleptic additive often precipitates as a solid or liquid emulsion, and as a result its bioavailability decreases. Furthermore, many compounds are not soluble in water-miscible, organic solvents. In another approach, lipophilic compounds are part of multiphase emulsions containing oils and solvents in combination with surfactants. These compositions may improve the bioavailability, but do not significantly increase the solubility of a lipophilic compound in aqueous media, and are usually used in topical applications only. Another technology uses vitamin E, or a sterol attached to hydrophilic moieties as a solubilizing agent for lipophilic compounds (U.S. Pat. No. 6,632,443 to Borowy-Borowski et al.).

Other potentially interesting materials for the preparation of water-insoluble flavor microparticles are salts of anionic polysaccharides such as the calcium salts of alginic acid, pectin and gellan gum. Calcium alginate, in particular, has found useful application as a water insoluble matrix for the encapsulation of microbial cells (T. Shiotani and T. Yamane, Eur. J. Appl. Microbiol. Biotechnol. 13 (2)96-101 [1981], H. C. Provost, Divies and T. Rousseau, Biotechnol. Lett. 7 (4)247-52 [1985]), enzymes (P. Brodelius and K. Mosbach, Adv. Appl. Microbiol. 28, 1 [1982]), drugs (H. Tomida, C. Mizuo, C. Nakamura and S. Kiryu, Chem. Pharm. Bull. 41(12)2161-2165 [1993]), vitamins (U.S. Pat. No. 4,389,419), colorings (K. Saito, T. Mori and K. I. Miyamoto, Food Chem. 50, 311-312 [1994]), and herbicides (A. B. Pepperman, J. C. W. Kuan and C. McCombs, J. Controlled Release 17, 105 [1991]).

The use of alginate for controlled flavor delivery is described in European patent application 221,850. According to this encapsulation in calcium alginate is used for controlled delivery of water-insoluble flavoring agents from chewing gum. The process for encapsulation involves separation of the alginate matrix from a large excess of water followed by air drying. Therefore, this process is not suitable for encapsulation of water-soluble and volatile flavoring agents, because these compounds either remain in the aqueous phase or volatilize during drying. Moreover, the approach does not allow control of flavor release by variation of particle size, porosity and flavor solvent composition.

The present invention provides a new method of solubilizing hydrophobic compounds, thereby enhancing their organoleptic properties, which overcomes many prior art drawbacks and limitations.

### SUMMARY OF THE INVENTION

In a first aspect, the present invention provides a method of enhancing an organoleptic property of a composition (e.g., an aqueous composition) that includes an organoleptic additive. In one example, the organoleptic property is a member selected from flavor, fragrance and combinations thereof. Exemplary organoleptic additives are lipophilic flavoring agents, fragrance additives and combinations thereof. The method includes solubilizing the additive (e.g., by forming an emulsion of said additive) in an aqueous carrier using a solubilizing agent of the invention, e.g., a solubilizing agent according to Formulae (I) to (VII).

According to a second aspect, the present invention provides a water-soluble composition including a solubilizing agent of the invention, e.g., a solubilizing agent according to Formulae (I) to (VII), and an organoleptic additive (e.g., a flavoring agent or fragrance of the invention), such that the composition has a detectably enhanced organoleptic property (e.g., flavor or fragrance) when compared to an essentially identical composition in which the solubilizing agent is not present or is present in a lesser amount than that present in a composition of the invention.

In another aspect, the invention provides a method comprising contacting an organoleptic additive and a solubilizing agent of the invention, e.g., a solubilizing agent according to Formulae (I) to (VII), with a water-based carrier forming a composition, wherein said organoleptic additive has a decreased vapor pressure, relative to an essentially identical composition wherein said solubilizing agent is not present.

In yet another aspect, the invention provides a method of preserving an organoleptic property of an organoleptic additive in a water-based carrier. An exemplary method includes: (a) mixing the organoleptic additive and a solubilizing agent of the invention, e.g., a solubilizing agent according to Formulae (I) to (VII), thereby forming a mixture; and (b) contacting said mixture with said water-based carrier.

In another aspect, the invention provides a composition formed by a method comprising: (a) combining an organoleptic additive (e.g., a fragrance, a flavoring agent and combinations thereof) with a solubilizing agent of the invention (e.g., PTS), thereby forming an additive-solubilizing agent mixture; and (b) contacting the additive-solubilizing agent mixture with a water-based carrier (e.g., water).

A method comprising: contacting an organoleptic additive and a solubilizing agent having a structure according to Formula (I):

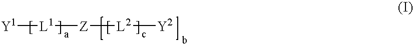

wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl,
   with a water-based carrier forming a composition, wherein said organoleptic additive has a decreased vapor pressure, relative to an essentially identical composition wherein said solubilizing agent is not present

Additional aspects, embodiments and objects of the present invention are set forth in the detailed description that follows.

### BRIEF DESCRIPTION OF THE DRAWINGS

FIG. 1 is a list of exemplary natural flavoring agents useful in the compositions and methods of the invention.

FIG. 2 is a list of synthetic and nature-identical flavoring agents useful in the compositions and methods of the invention.

### DETAILED DESCRIPTION OF THE PREFERRED EMBODIMENTS

### Definitions and Abbreviations

Unless defined otherwise, all technical and scientific terms used herein generally have the same meaning as commonly understood by one of ordinary skill in the art to which this invention belongs. The nomenclature used herein and the laboratory procedures in analytical chemistry, and organic synthesis described below are those well known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl subsituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The solubilized organoleptic additives provided by the invention can be used for the aromatization or flavoring of foodstuffs, beverages, pharmaceuticals, chewing-gums, oral hygiene products (e.g. toothpaste) or other healthcare (e.g., skin care) products.

The term “beverage” describes any water-based liquid, which is suitable for human consumption (i.e., food-grade). “Beverage” can be any commonly available beverage (e.g., any marketed beverage). The term “beverage” includes beers, carbonated and non-carbonated waters (e.g., table waters and mineral waters), flavored waters (e.g., fruit-flavored waters), mineralized waters and other fortified waters, sports drinks (e.g., Gatorade), smoothies, neutraceutical drinks, filtered or non-filtered fruit and vegetable juices (e.g., apple juice, orange juice, cranberry juice, pineapple juice, lemonades and combinations thereof) including those juices prepared from concentrates, and cocktails or mixtures of any of the above listed beverages. Exemplary juices include fruit juices having 100% fruit juice (squeezed or made from concentrate), fruit drinks (e.g., 0-29% juice), nectars (e.g., 30-99% juice). The term “beverage” also includes fruit flavored beverages, carbonated drinks, such as soft-drinks, fruit-flavored carbonates and mixers. Soft drinks include caffeinated soft drinks, such as coke (e.g., Pepsi Cola, Coca Cola) and any “diet” versions thereof (e.g., including non-sugar sweeteners). The term “beverage” also includes teas (e.g., green and black teas, herbal teas) including instant teas, coffee, including instant coffee, chocolate-based drinks, malt-based drinks, milk, drinkable dairy products and beer. The term “beverage” also includes any liquid or powdered concentrates used to make beverages (e.g., frozen and shelf-stable).

The term “non-alcoholic beverage” includes beverages containing essentially no alcohol. Exemplary non-alcoholic beverages include those listed above for the term “beverage”. The term “non-alcoholic beverage” includes beers, including those generally referred to as “non-alcoholic beers”. In one example, the non-alcoholic beverage includes less than about 10% alcohol by volume. In another example, the non-alcoholic beverage includes less than about 9% or less than about 8% alcohol by volume. In yet another example, the non-alcoholic beverage includes less than about 7%, less than about 6% or less than about 5% alcohol by volume.

The term “aqueous” and “water-based” are used interchangeably herein and means a composition containing at least 50%, at least 60%, at least 70%, at least 80%, at least 90%, at least 95%, at least 98% (w/w) water or more than 98% (w/w) water.

The term “water-soluble” when referring to a formulation or compositions of the invention, means that the composition when added to an aqueous medium (e.g., water, original beverage) dissolves in the aqueous medium to produce a solution that is essentially clear. In one example, the formulation dissolves in the aqueous medium without heating the resulting mixture above ambient temperature (e.g., 25° C.). The term “essentially clear” is defined herein.

The term “essentially clear” is used herein to describe the compositions (e.g., formulations) of the invention. For example, the term “essentially clear” is used to describe an aqueous formulation or a beverage of the invention. In one example, clarity is assessed by the normal human eye. In this example, “essentially clear” means that the composition is transparent and essentially free of visible particles and/or precipitation (e.g., not visibly cloudy, hazy or otherwise non-homogenous). In another example, clarity, haziness or cloudiness of a composition is assessed using light scattering technology, such as dynamic light scattering (DLS), which is useful to measure the sizes of particles, e.g., micelles, contained in a composition. In one example, “essentially clear” means that the median particle size as measured by DLS is less than about 100 nm. For example, when the median particle size is less than 100 nm the liquid appears clear to the human eye. In another example, “essentially clear” means that the median particle size is less than about 80 nm. In yet another example, “essentially clear” means that the median particle size is less than about 60 nm. In a further example, “essentially clear” means that the median particle size is less than about 40 nm. In another example, “essentially clear” means that the median particle size is between about 20 and about 30 nm. A person of skill in the art will know how to prepare a sample for DLS measurement. For example, in order to prepare a sample (e.g., formulation of the invention) for a DLS measurement, the sample is typically diluted so that the concentration of the solubilizing agent in the diluted sample is between about 1 mM (10⁻³ M) and 0.01 mM (10⁻⁵ M). In another example, the solubilizing agent (e.g., PTS) is present in a concentration that is above the critical micelle concentration (CMC) (i.e., concentration that allows for spontaneous formation of micelles). For example, a typical CMC for PTS in water is about 0.1 to about 0.5 mg/ml. A person of skill in the art will be able to select suitable concentrations in order to successfully measure particle sizes in a formulation of the invention.

Alternatively, clarity, haziness or cloudiness of a composition of the invention can be determined by measuring the turbidity of the sample. This is especially useful when the composition is a beverage (e.g., water, soft-drink etc.). In one example, turbidity is measured in FTU (Formazin Turbidity Units) or FNU (Formazin Nephelometric Units). In one example, turbidity is measured using a nephelometer, known in the art. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the invention (e.g., a beverage of the invention) is “essentially clear” when the turbidity is not more than about 500% higher than the control (original beverage without an added lipophilic bioactive molecule of the invention, but optionally including a solubilizing agent of the invention, e.g. PTS). For example, the turbidity of a sample of flavored water is measured to be 2.0 ntu and the turbidity of another sample containing the same flavored water in combination with ubiquinol is measured to be at or below about 8.0 ntu (2.0 ntu+200%=8.0 ntu), then the ubiquinol sample is considered to be essentially clear. In another example, a composition of the invention is “essentially clear” when the turbidity is not more than about 300% higher than the control. In yet another example, a composition of the invention is “essentially clear” when the turbidity is not more than about 200%, about 150% or about 100% higher than the control. In a further example, a composition of the invention is “essentially clear” when the turbidity is not more than about 80%, about 60%, about 40%, about 20% or about 10% higher than the control.

The term “emulsion” as used herein refers to an organoleptic additive of the invention emulsified (solubilized) in an aqueous medium using a solubilizing agent of the invention. In one example, the emulsion includes micelles formed between the additive(s) and the solubilizing agent. When those micelles are sufficiently small, the emulsion is essentially clear. Typically, the emulsion will appear clear (e.g., transparent) to the normal human eye, when those micelles have a median particle size of less than 100 nm. In one example, the micelles in the emulsions of the invention have median particle sizes below 60 nm. In a typical example, micelles formed in an emulsion of the invention have a median particle size between about 20 and about 30 nm. In another example, the emulsion is stable, which means that separation between the aqueous phase and the organoleptic component does essentially not occur (e.g., the emulsion stays clear). A typical aqueous medium, which is used in the emulsions of the invention, is water, which may optionally contain other water-soluble (e.g., solubilized) molecules, such as salts, coloring agents, flavoring agents and the like. In one example, the aqueous medium of the emulsion does not include an alcoholic solvent, such as ethanol or methanol.

The term “micelle” is used herein according to its art-recognized meaning and includes all forms of micelles, including, for example, spherical micelles, cylindrical micelles, worm-like micelles and sheet-like micelles.

The term “tocopherol” includes all tocopherols, including alpha-, beta-, gamma- and delta tocopherol. The term “tocopherol” also includes tocotrienols.

The term “detergent” includes any soap-based and non-soap-based detergents, such as household cleaners (e.g., floor, window, general purpose cleaners), scouring and disinfection products, dish detergents, dishwasher detergents, soaps (e.g., hand-soaps), hair shampoos, bath and shower gels, hair conditioners and other personal cleansing products, laundry detergents, fabric washing powders, washing liquids, fabric softeners and other fabric care products.

In a generally preferred embodiment, the organoleptic property of one or more additives (e.g., flavor or fragrance) is enhanced by its combination with, and preferably solubilization by, in an aqueous carrier, a solubilizing agent for that additive, without a concomitant increase in the amount of the additive included in the composition comprising the solubilizing agent. In an exemplary embodiment, the flavor or fragrance perceived by an observer of a first composition with a first amount of additive, is less than that perceived by an observer of a second composition containing essentially the first amount of additive and a solubilizing agent, solubilizing the additive. Expressed another way, the second composition is perceived as having a second amount of the additive, wherein the perceived second amount is greater than the first amount.

The flavor or fragrance properties of the compositions may be used as such to impart, strengthen or improve the flavor or fragrance of a wide variety of products. For example, an additive solubilized by a solubilizing agent, as set forth herein, may be used as a component of a perfume (or fragrance composition) to contribute its fragrance character to the overall fragrance of such perfume or fragrance or taste of a food or beverage to enhance the flavor of the food or beverage. For the purposes of this invention a perfume is intended to mean a mixture of fragrance materials, if desired, mixed with or dissolved in a suitable solvent or solvents or mixed with a solid substrate (i.e., a carrier), which is used to impart a desired fragrance to the skin and/or product for which an agreeable fragrance is indispensable or desirable. Examples of such products are air fresheners, room sprays and pomanders; soaps, cosmetics such as creams, ointments, toilet waters, preshave, aftershave, skin and other lotions, talcum powders, body deodorants and antiperspirants, etc.

As used herein, the term “additive,” refers to an organoleptic species having a fragrance (e.g., a scent, aroma), and/or a taste (e.g., a flavor or feeling in the mouth). The additive is preferably part of a composition. Exemplary compositions include liquid compositions, semi-solid and solid compositions. In one example, the composition is a water-based (aqueous) composition. In another example, the additive is a component of a food, a beverage, a washing detergent, a skin-care product, or other scented product.

### Additives

Additives which can be advantageously combined with one or more solubilizing agents to enhance an organoleptic property according to the invention in a perfume, beverage or food are, for example, natural products such as extracts, essential oils, absolutes, resinoids, resins, concretes etc., but also synthetic materials such as hydrocarbons, alcohols, aldehydes, ketones, ethers, acids, esters, acetals, ketals, nitrites, etc., including saturated and unsaturated compounds, aliphatic, carbocyclic, and heterocyclic compounds.

Such fragrance and flavoring agents are mentioned, for example, in S. Arctander, Perfume and Flavor Chemicals (Montclair, N.J., 1969), in S. Arctander, Perfume and Flavor Materials of Natural Origin (Elizabeth, N.J., 1960), in “Flavor and Fragrance Materials—1991”, Allured Publishing Co. Wheaton, Ill. USA and the database maintained by the Research Institute for Fragrance Materials. (http://rifm.org/about_rifm.htm). Each of these references is incorporated herein by reference in their entirety for all purposes.

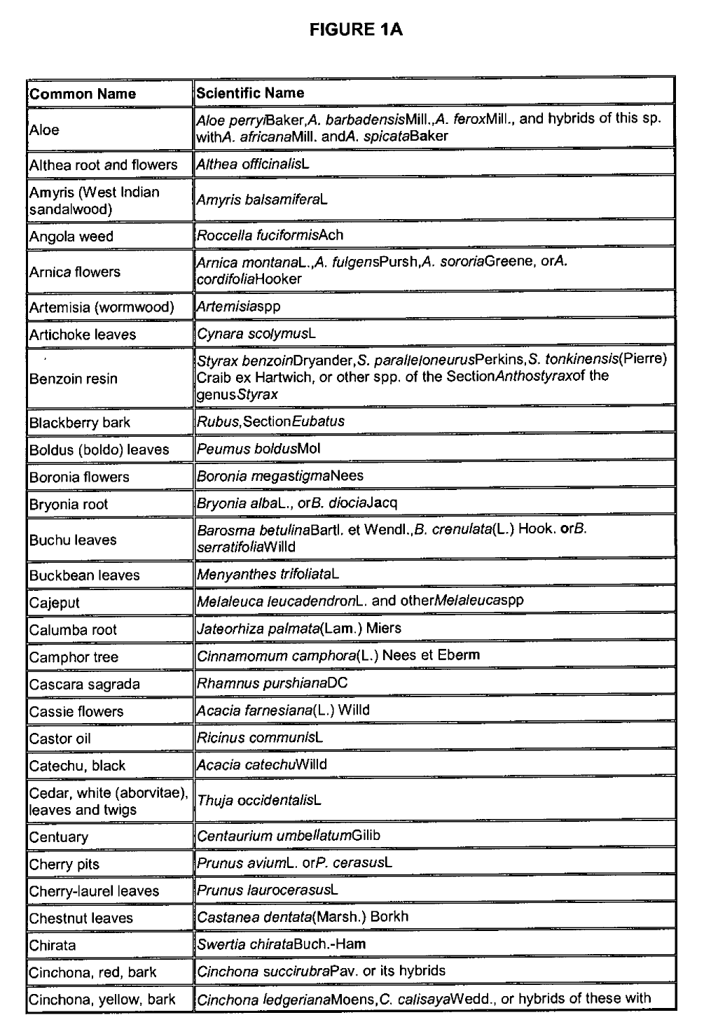

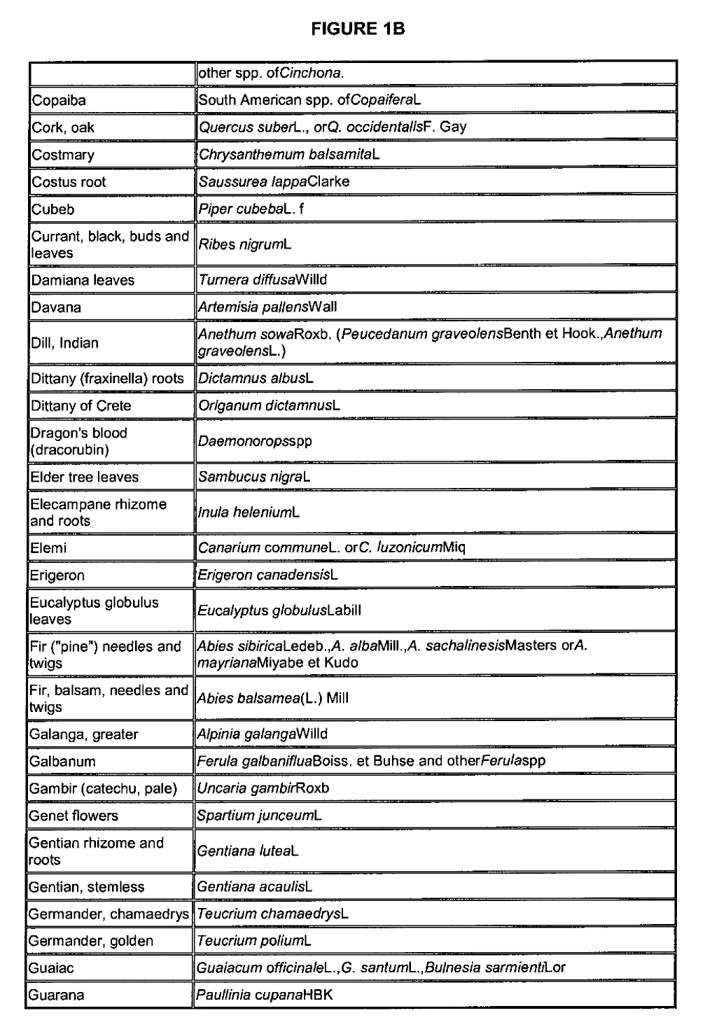

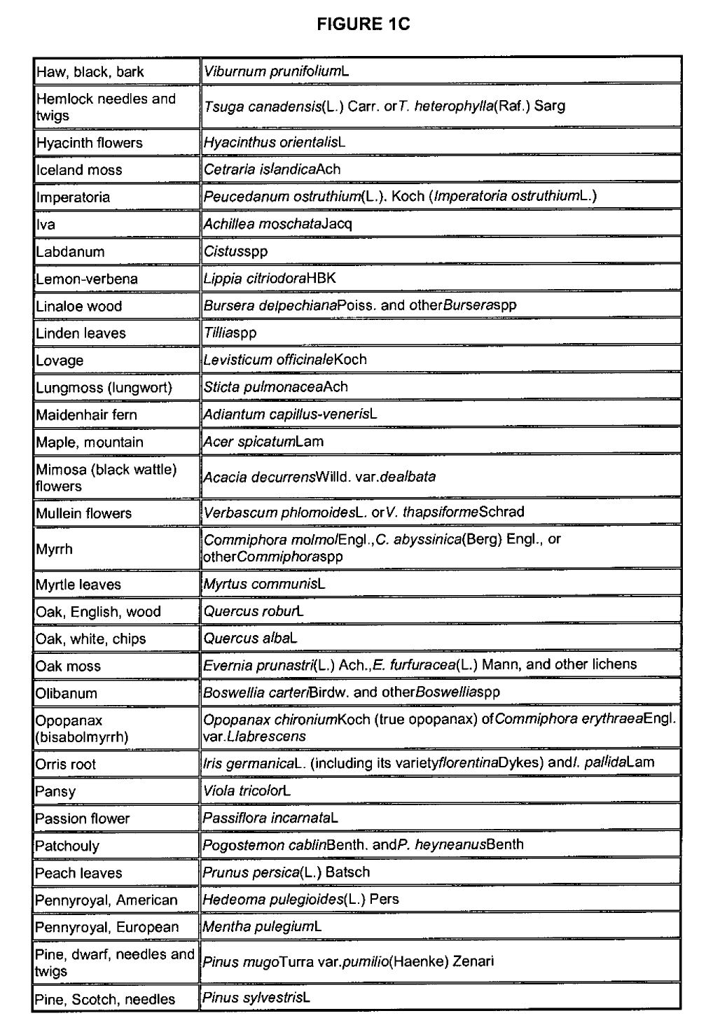

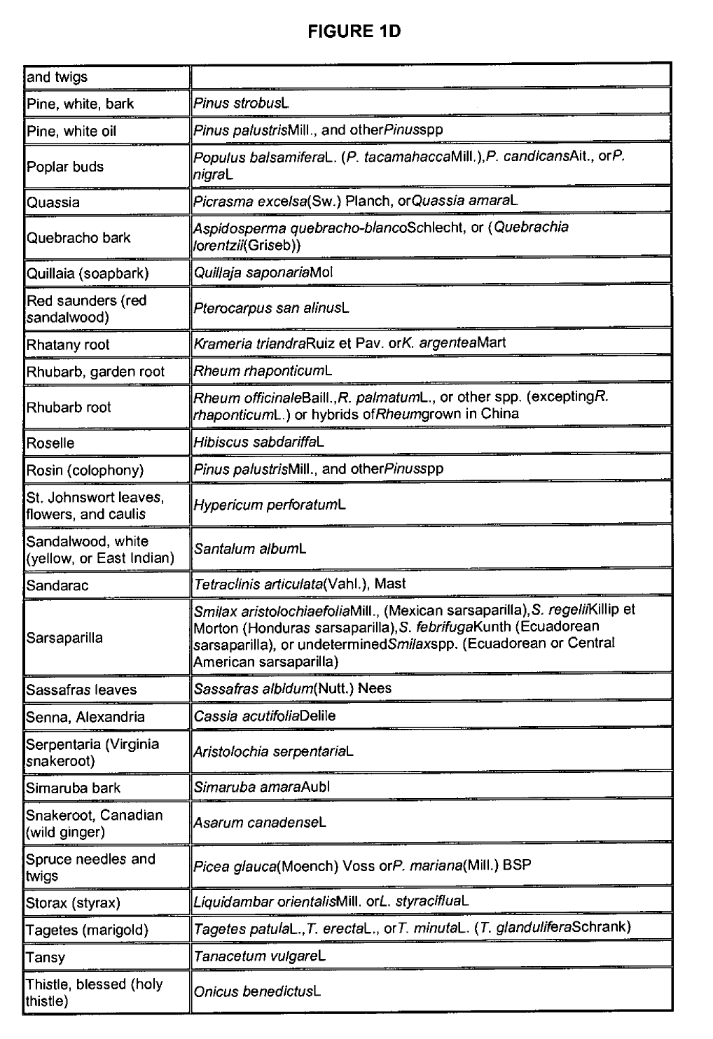

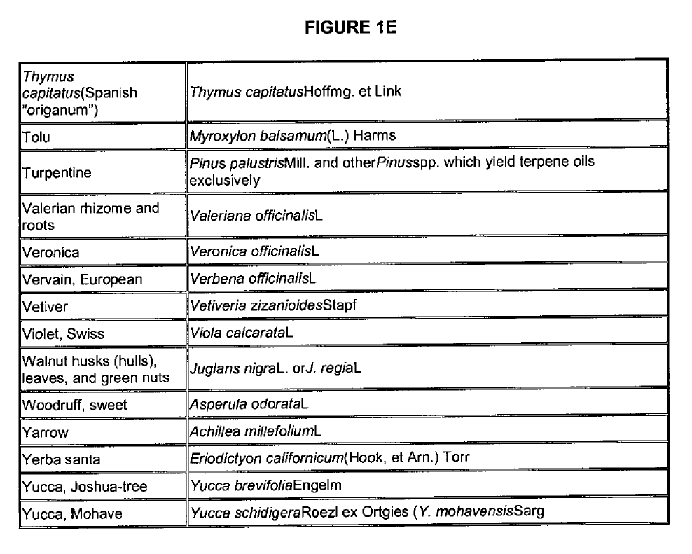

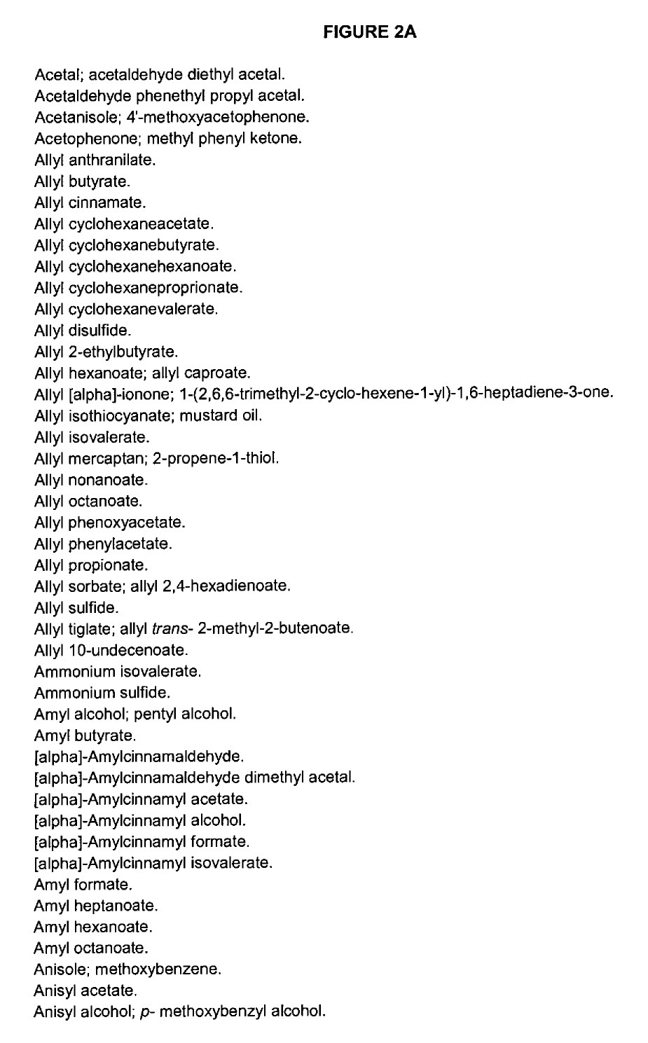

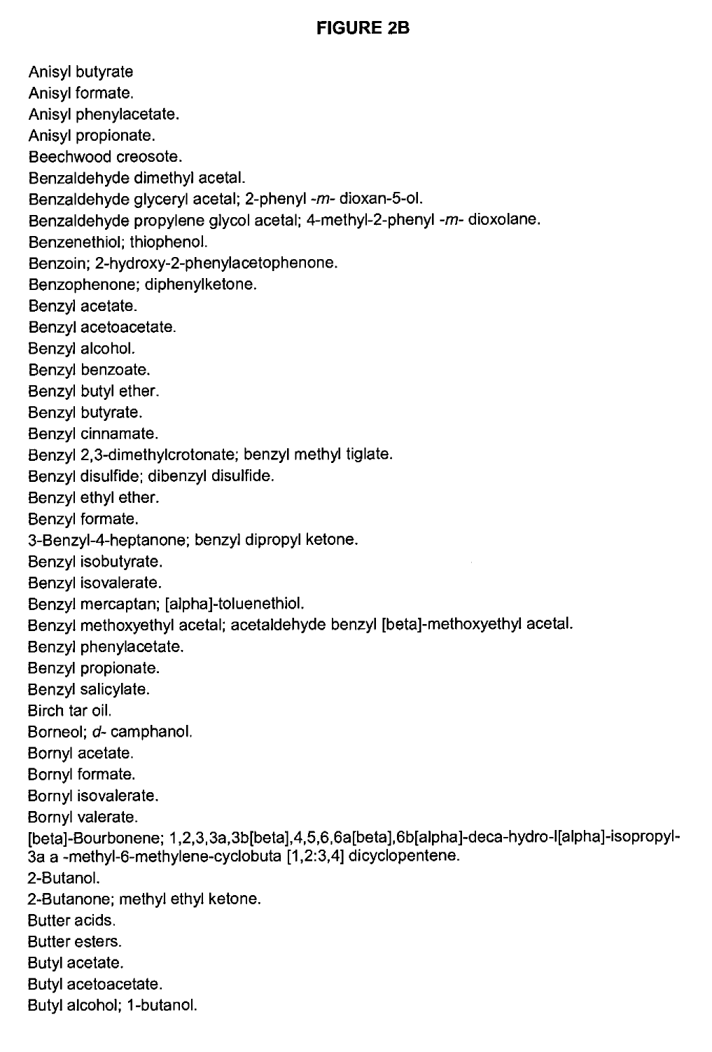

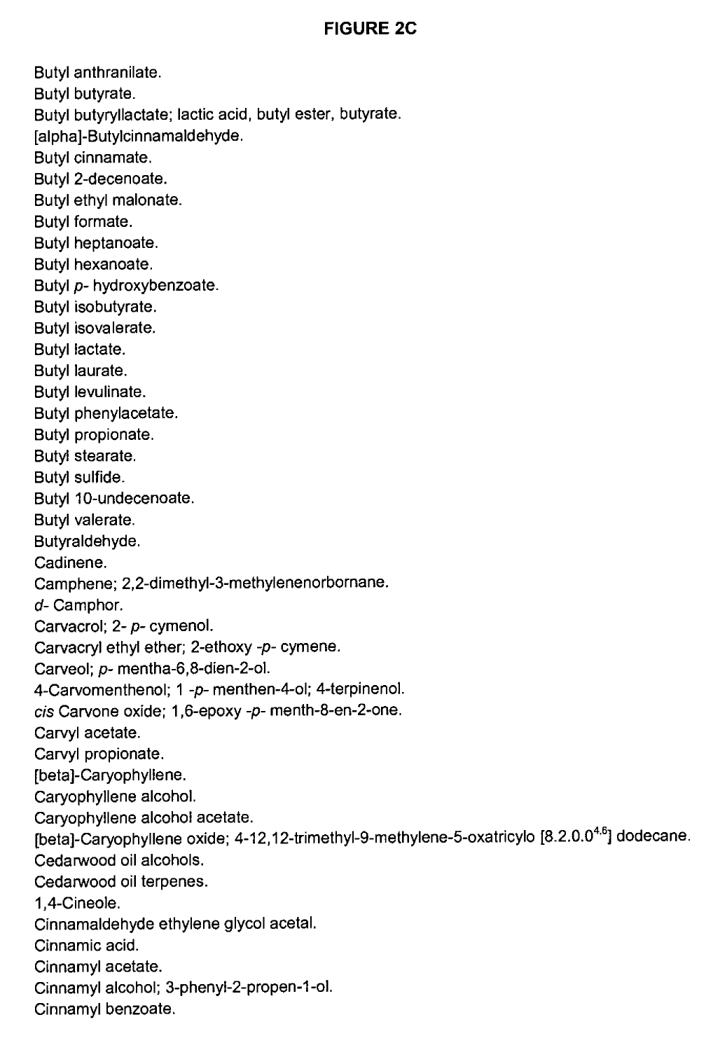

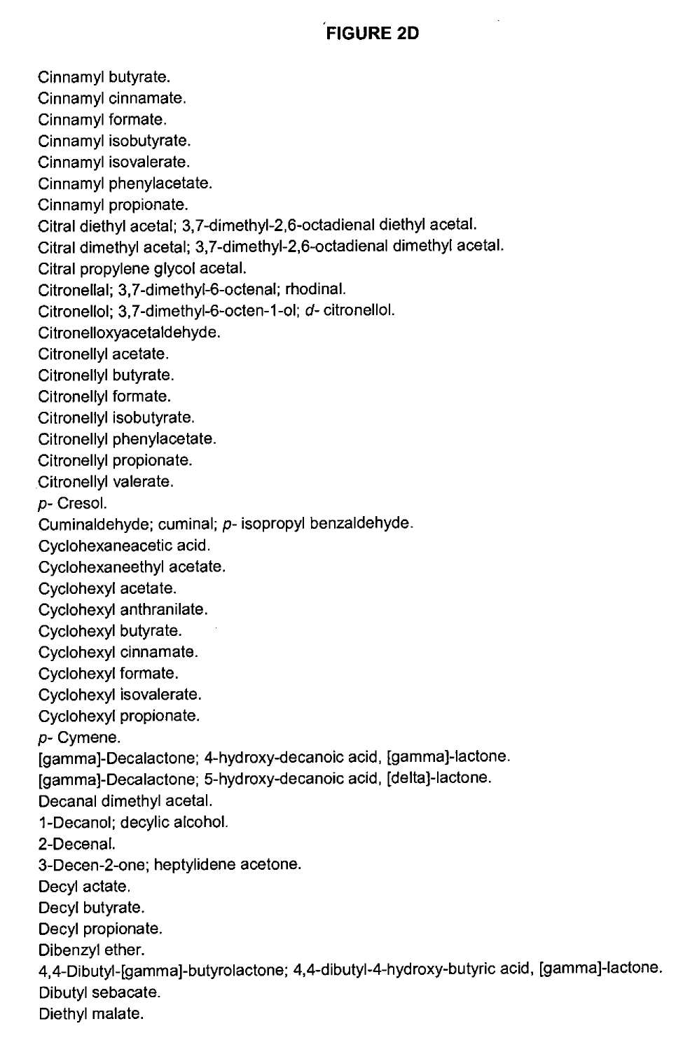

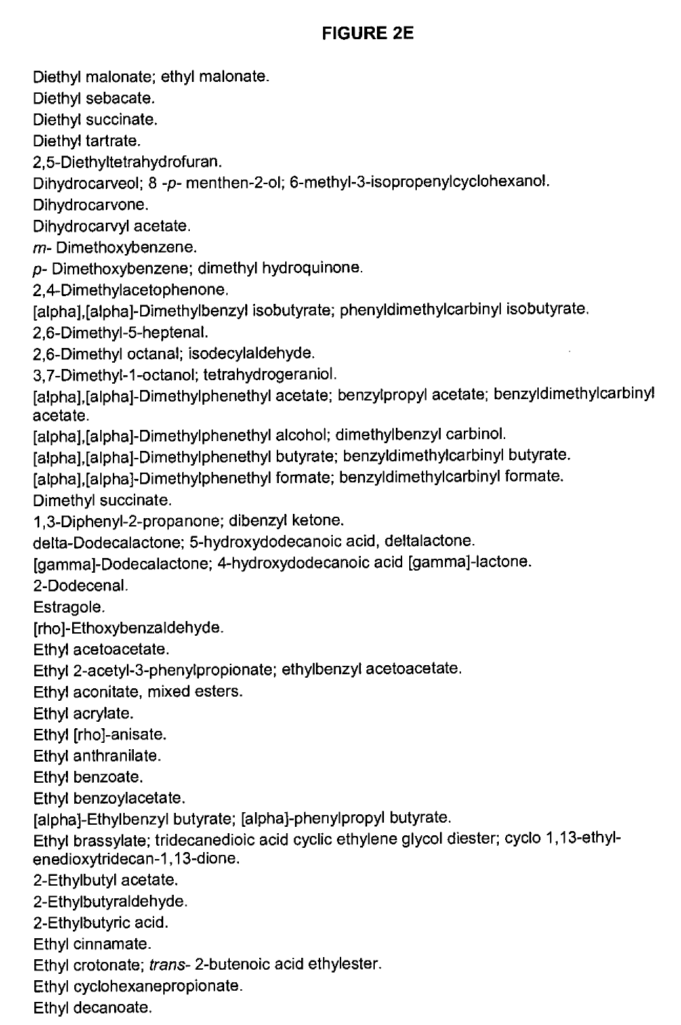

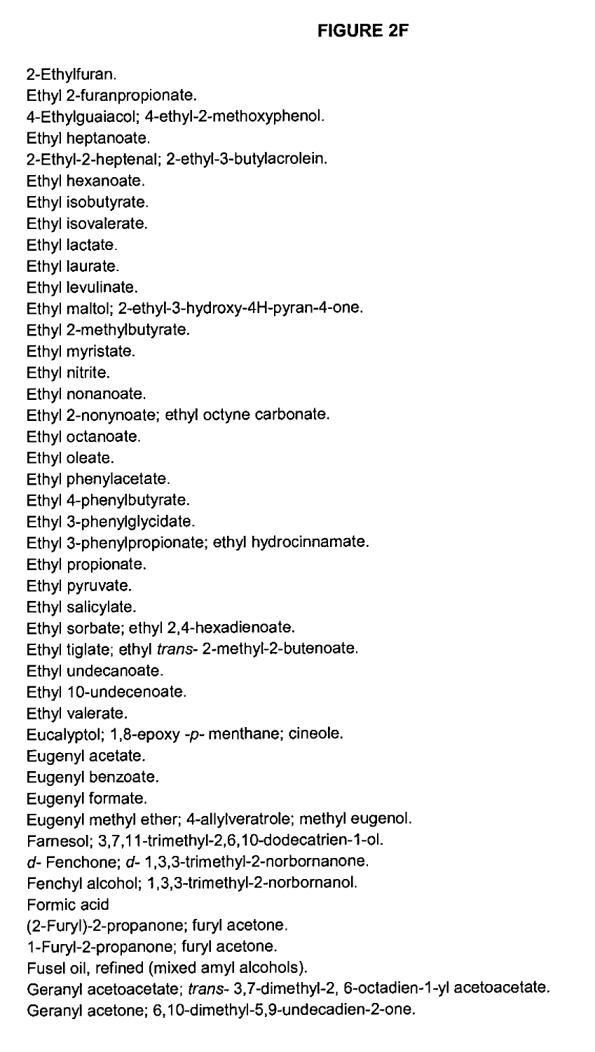

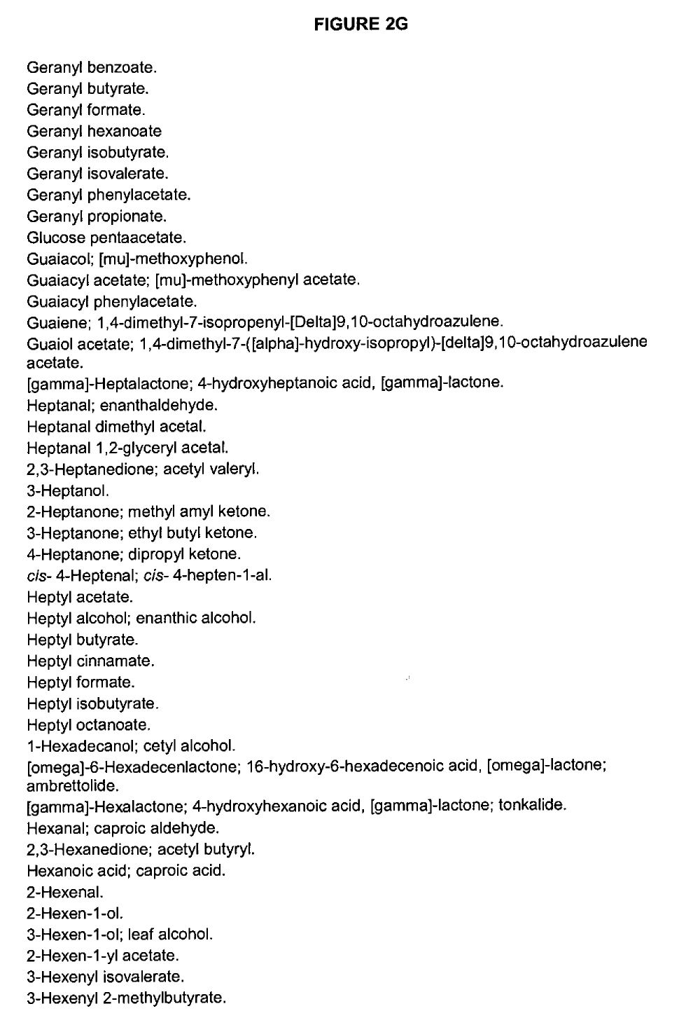

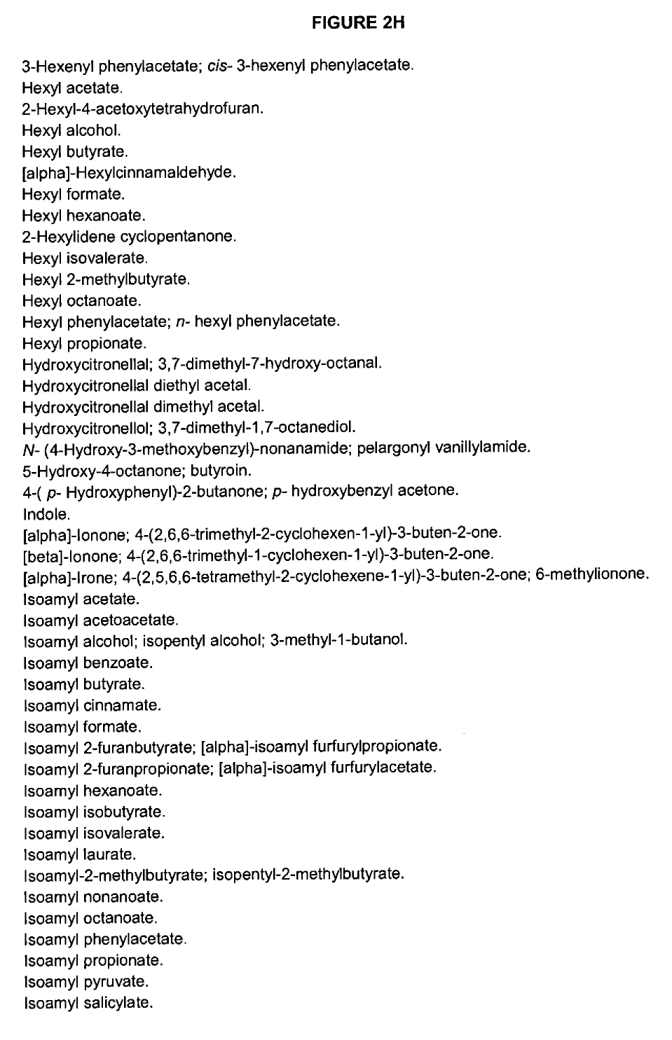

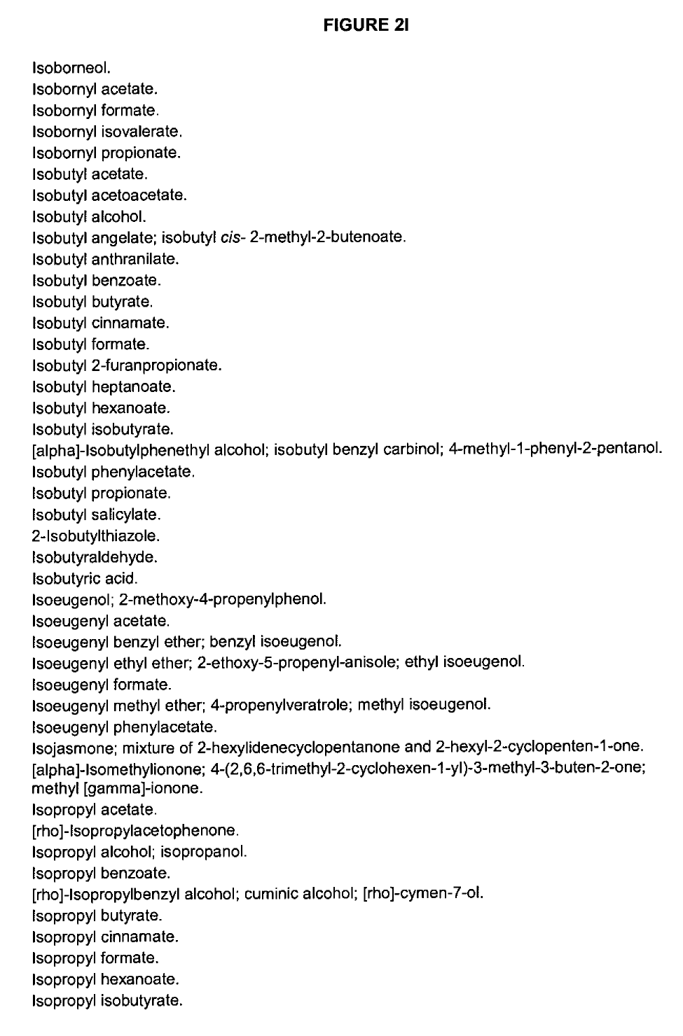

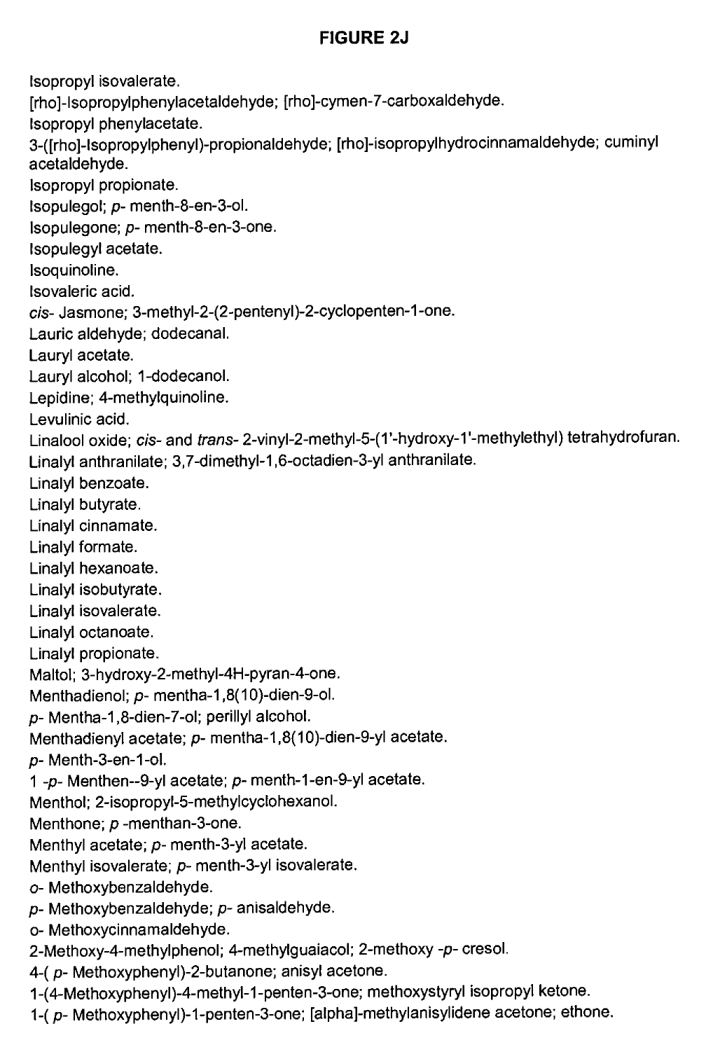

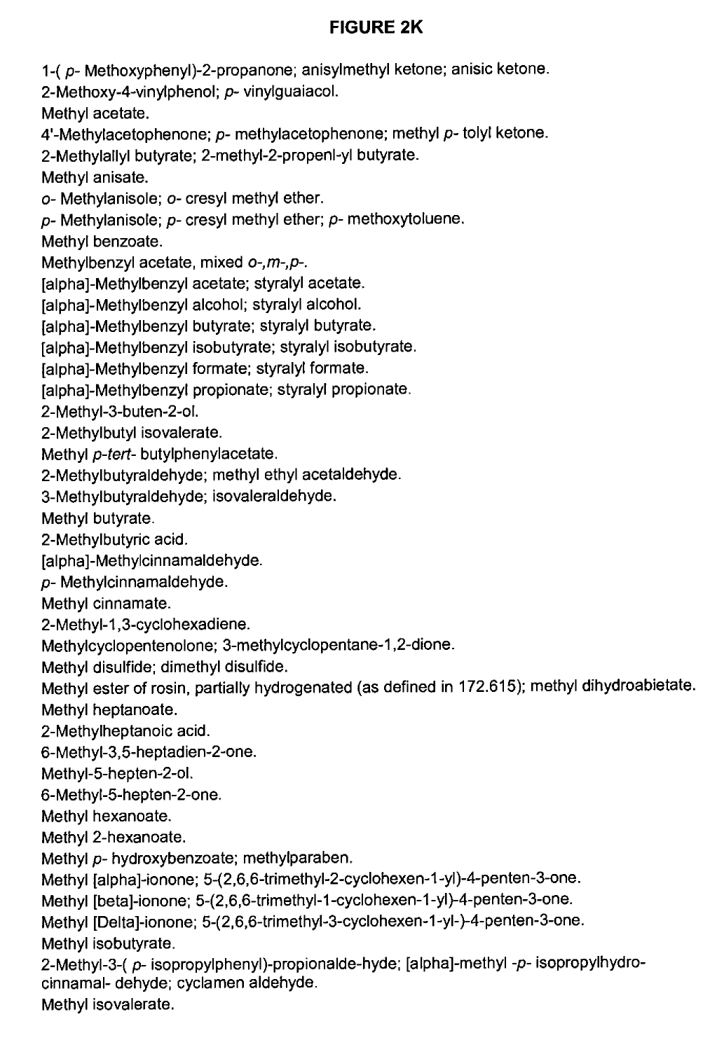

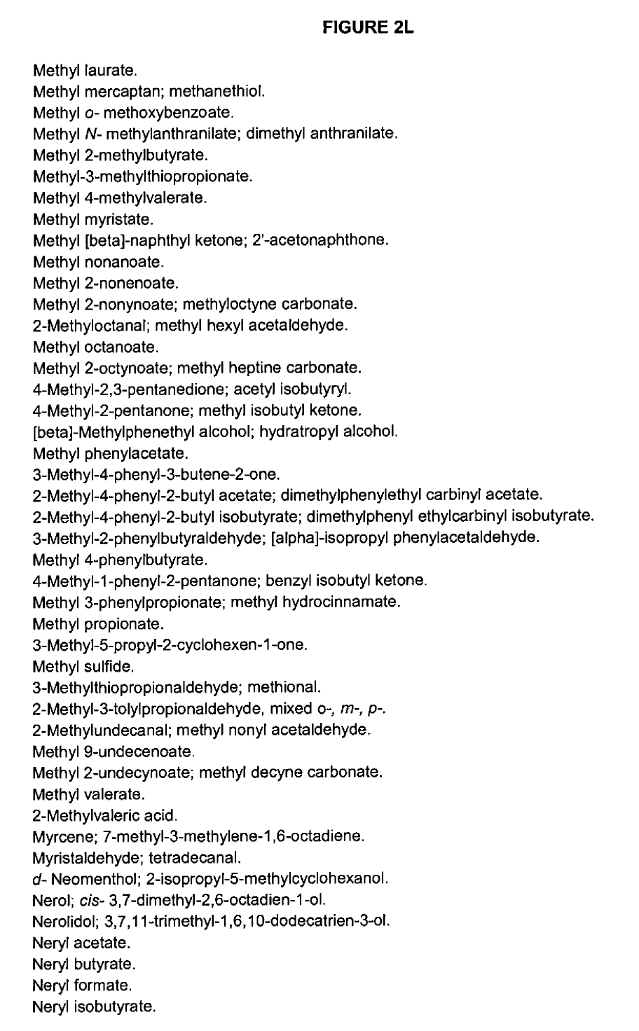

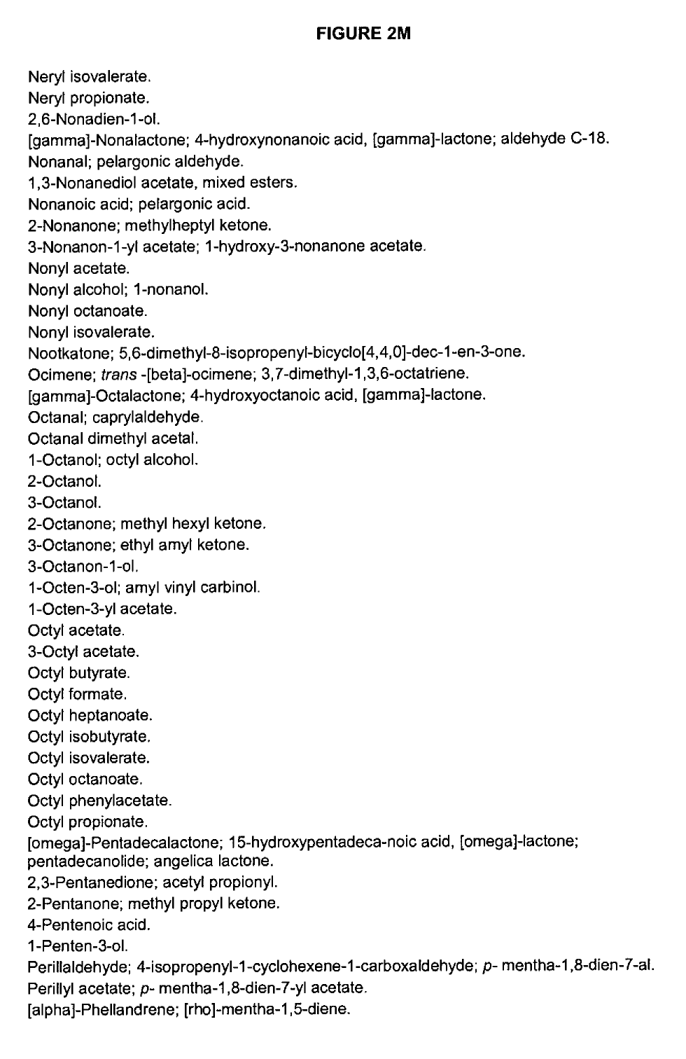

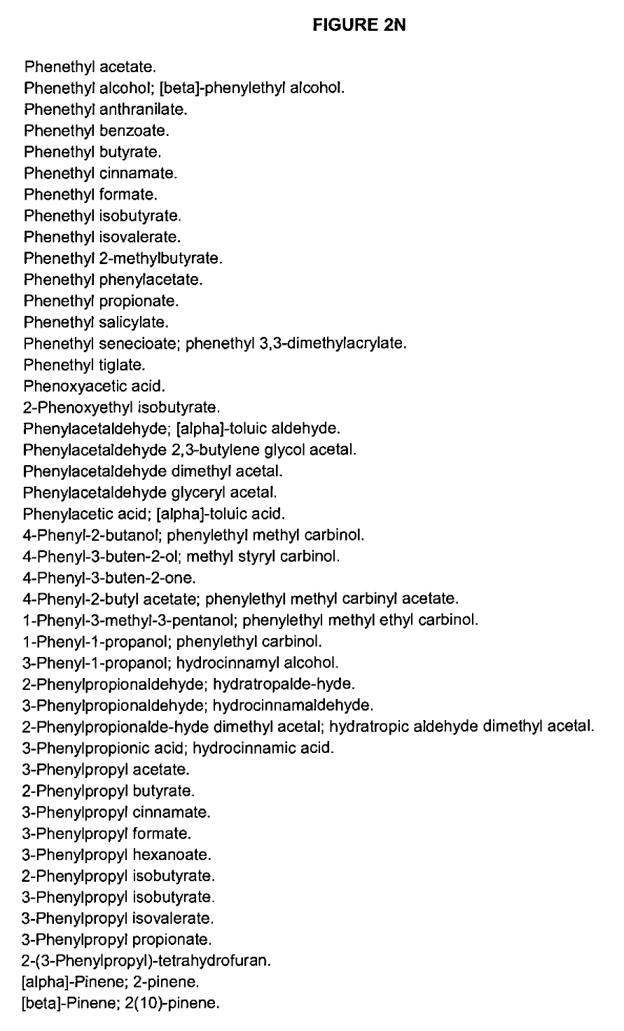

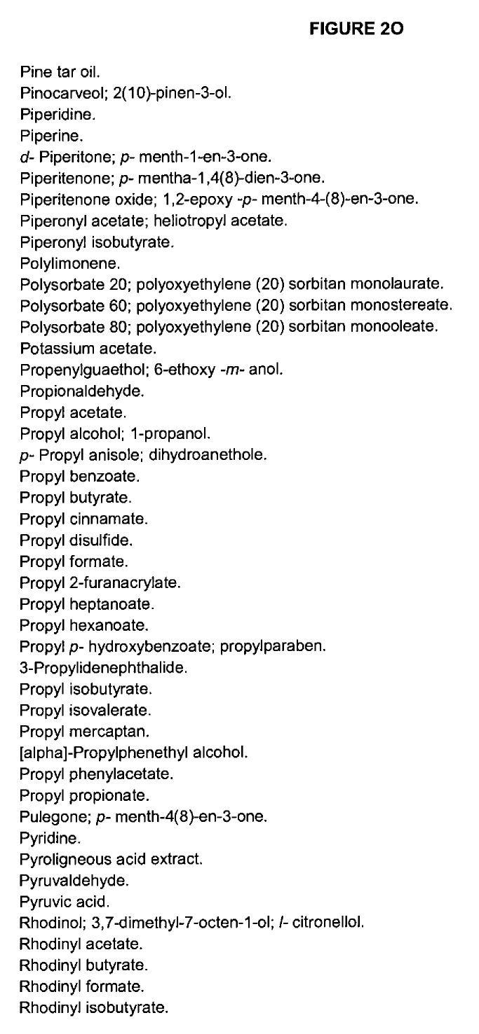

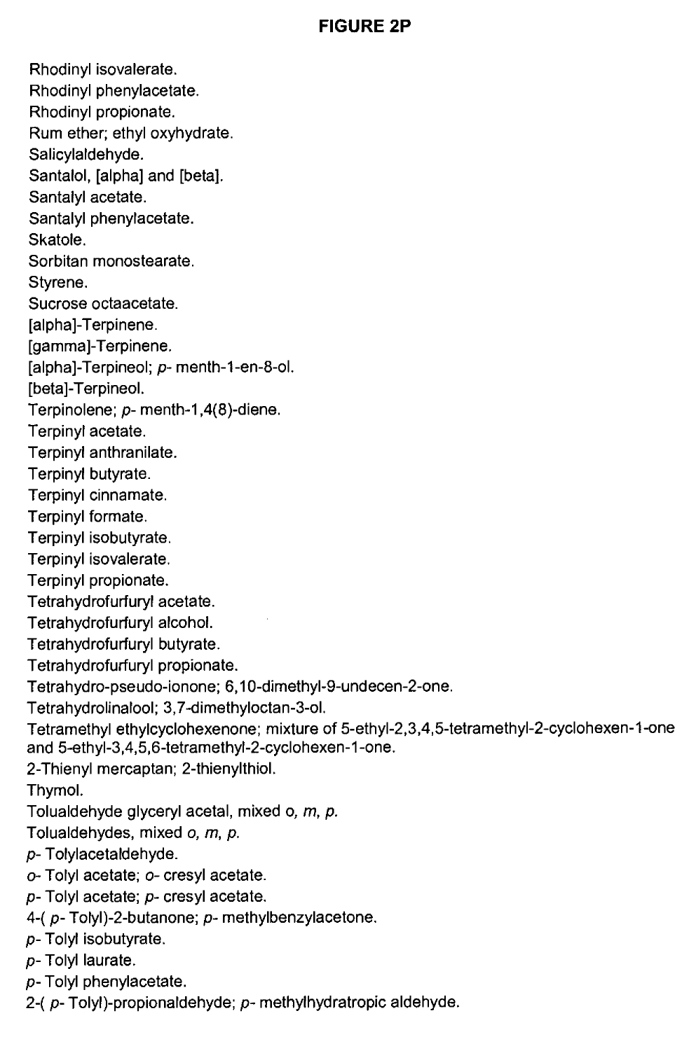

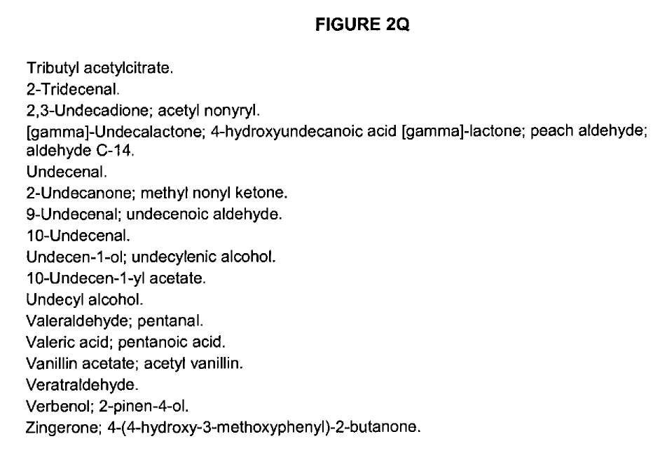

Exemplary flavoring agents include those approved by the U.S. Food and Drug Administration (FDA) for human consumption. In one example, the flavoring agent is selected from natural, nature-identical (e.g., not artificial) and artificial (e.g., synthetic) flavoring agents. In another example, the flavoring agent is an extract of natural origin (e.g., flavoring preparation) or a smoke flavoring. In yet another example, the flavoring agent is an essence or extract obtained from plants listed in U.S. Code of Federal Regulations, Title 21 (21 CFR) §§182.10, 182.20, 182.40, and 182.50, 184, and the substances listed in §172.510 and §172.515. Exemplary natural, nature-identical and artificial flavoring agents are listed in FIGS. 1 and 2. In one example, the additive is not a terpene. In another example, the additive is not a terpenoid. In yet another example, the additive is not a compound having a high content of polyunsaturated fatty acids.

In an exemplary embodiment, the additive is an oil or an oil component. The term “oil” includes oils derived from plant material, such as seed oils and essential oils. In one example, the oil is of food grade. Exemplary essential oils include citrus oils, bergamot oil, jasmine oil, ylang ylang oil, rosemary oil, cinnamon oil, lavender oil, rose oil, rose geranium oil, patchouli oil, neroli oil, vetiver oil and the like. The term essential oil also includes fragrances and flavoring oils (e.g., fruit flavor oils, citrus flavor, almond flavor).

Examples of additives include molecules associated with seeds (e.g., caraway, anise, sesame, etc.); woods (e.g., oak, beech maple (hard, soft, sugar), birch, teak) and fruitwoods (e.g., pecan, apple, peach, pear, apricot, cherry, walnut). Wood-based flavoring agents include versions of the same wood that have been toasted to varying degrees, charred or charcoaled. Other additives are derived from nuts (e.g., pecan, walnut, almond, cashew, hazelnut, macadamia, coconut); fruits (e.g., apricot, apple, cherry, citrus (lemon, lime, grapefruit, tangerine, tangelo, cumquat, etc.), grape, raisin, mango, pineapple, plum); herbs, vegetables, spices and other plant parts (e.g., mints, vanilla, cinnamon, cocoa, peppers, artichoke, celery, etc.).

Additional exemplary additives include geraniol, geranyl acetate, linalol, linalyl acetate, tetrahydrolinalol, citronellol, citronellyl acetate, dihydromyrcenol, dihydromyrcenyl acetate, tetrahydromyrcenol, terpineol, terpinyl acetate, nopol, nopyl acetate, 2-phenyl-ethanol, 2-phenylethyl acetate, benzyl alcohol, benzyl acetate, benzyl salicylate, styrallyl acetate, benzyl benzoate, amyl salicylate, dimethylbenzyl-carbinol, trichloromethylphenyl-carbinyl acetate, p-tert-butylcyclohexyl acetate, isononyl acetate, vetiveryl acetate, vetiverol, α-hexylcinnamaldehyde, 2-methyl-3-(p-tert-butylphenyl)propanal, 2-methyl-3-(p-isopropylphenyl)propanal, 2-(p-tert-butylphenyl)-propanal, 2,4-dimethyl-cyclohex-3-enyl-carboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyl-tetrahydropyran, 3-carboxymethyl-2-pentylcyclopentane, 2-n-heptylcyclopentanone, 3-methyl-2-pentyl-2-cyclopentenone, n-decanal, n-dodecanal, 9-decenol-1, phenoxyethyl isobutyrate, phenylacetaldehyde dimethylacetal, phenylacetaldehyde diethylacetal, geranyl nitrile, citronellyl nitrile, cedryl acetate, 3-isocamphylcyclohexanol, cedryl methyl ether, isolongifolanone, aubepine nitrile, aubepine, heliotropin, coumarin, eugenol, vanillin, diphenyl oxide, hydroxycitronellal, ionones, methylionones, isomethylionones, irones, cis-3-hexenol and esters thereof, indan musks, tetralin musks, isochroman musks, macrocyclic ketones, macrolactone musks, ethylene brassylate, ellagic acid, gallic acid, and syringaldehyde.

The invention includes solubilization of organolpetics in water or aqueous (water-based) mixtures that are combinations of additives, e.g., flavorings for soft drinks, colas, etc.

The invention further include solubilization of organoleptics in a water-based or mixed solvent system carrier that is carbonated, includes phosphorus-based acids (and salts thereof), and combinations thereof.

The organoleptic enhancement of additives solubilized as set forth herein includes enhancement of the effect of sweeteners and flavor blockers as well. For example, the invention provides for the enhancement of the organoleptic properties of the sweetener or blocker itself by solubilization with a solubilizing agent, or enhancement of the organoleptic properties of one or more additives other than the sweetener or blocker, giving the impression that there is more sweetener or blocker present in the solubilized formulation than in an essentially identical formulation that does not include the solubilizing agent.

By the term “sweetener,” as used herein, is meant any material which gives a sweet perception, including both high and low intensity sweeteners, e.g.,
A. monosaccharides, including but not limited to aldoses and ketoses beginning with trioses, including but not limited to glucose, galactose, and fructose;
B. compounds generically known as sugars, which include but are not limited to mono-, di- and oligosaccharides including but not limited to sucrose, maltose, lactose, etc.;
C. sugar alcohols which include but are not limited to sorbitol, mannitol, glycerol;
D. carbohydrates and polysaccharides which include but are not limited to polydextrose and maltodextrin;
E. high intensity sweeteners.

As used herein, “high intensity sweeteners,” includes, but is not limited to, L-aspartyl-L-phenylalanine methyl ester (Aspartame™) and other related dipeptide sweeteners, saccharin, L-aspartyl-D-alanine-N-(2,2,4,4-tetramethyl thiatan-3-yl) amide (Alitame™), 1,6-dichloro-1,6-dideoxy-β.-D-fructofuranoysl-4-chloro-4-deoxy-α-D-galactopyranoside (Sucralose™), 6-methyl-1,2,3-oxathiazin-4(3H)-one 2,2-dioxide (Acesulfame™), 6-methyl-1,2,3-oxathiazin-4(3H)-one 2,2-dioxide potassium salt (Acesulfame-K™), cyclohexylsulfamic acid (Cyclamate), N-(L-aspartyl)-N′(2,2,5,5,tetramethylcyclopentanoyl) 1,1-diaminoethane and its related compounds, guanidinium class sweeteners, dihydrochalcone class sweeteners, stevioside, miraculin and thaumatin, and their physiologically acceptable salts. Many more sweeteners are described in the following publications: Walters, D. E., Orthoefer, F. T., and DuBois, G. E., (Ed.), “Sweeteners Discovery, and Molecular Design, and Chemoreception,” ACS Symposium Series 450, American Chemical Society, Washington, D.C., 1991; Grenby, T. H., “Progress in Sweeteners,” Elsevier Applied Science Series, Elsevier Science Publishing, London and New York, 1989.

By the term “low intensity sweetener” is meant any sweetener except a high intensity sweetener.

By the term “blocker” or “masking agent” is meant any flavorful eatable which is used to cover and/or disguise and/or obscure an undesirable taste. Exemplary masking agents include sweeteners and spices such as onion, garlic, paprika, red pepper, chili powder, etc.

An exemplary blocker is a bitterness blocker. Suitable bitterness blockers include, for example, nucleotides such as those described in, for example, WO 00/38536 (Margolskee et al.); WO 02/096464A1 (McGregor et al.); U.S. 2002/0177576 (McGregor et al.); and U.S. Pat. No. 6,540,978 (Margolskee et al.). A class of naturally occurring compounds that can block the transduction of bitter taste by interrupting the process at several points is also described by Ming et. al. (Ding Ming et al., Blocking taste receptor activation of gustducin inhibits gustatory responses to bitter compounds, Proc. Natl. Acad. Sci., August, 1999, 9903-9908, vol. 96, USA). In one embodiment, the bitterness inhibitor is a monophosphate, such as adenosine monophosphate.

Other exemplary bitterness inhibitors include, for example, nucleotides (i.e., phosphate esters of nucleosides or nucleoside derivatives, and salts thereof) (e.g., sodium salts, disodium salts, potassium salts, dipotassium salts, lithium salts, ammonium salts, diammonium salts, alkylammonium salts, tris salts, and combinations thereof), and/or hydrates thereof. Preferred nucleotides include, for example, phosphate esters of ribonucleosides (e.g., adenosine, guanosine, cytidine, and uridine). More preferred nucleotides include phosphate esters of adenosine and phosphate esters of uridine. Exemplary phosphate esters include monophosphate esters (e.g., cyclic or non-cyclic), diphosphate esters, and combinations thereof. Suitable nucleotide monophosphate esters include, for example, 3-monophosphate esters, 5-monophosphate esters, and 3′,5′-cyclic monophosphate esters.

The quantities in which one or more additives (e.g., solubilized additives) are added to a composition, e.g., perfumes or in products to be perfumed may vary within wide limits and depend, inter alia, on the nature of the product, on the nature and the quantity of the other components of the perfume in which the amide is used and on the olfactive effect desired. It is therefore only possible to specify wide limits, which, however, provide sufficient information for the specialist in the art to be able to use a solubilized additive according to the invention for a specific purpose.

### Compositions

In one example, the invention provides compositions that include an organoleptic additive in combination with a solubilizing agent of the invention, e.g., those of Formulae (I) to (VII).

In another example, the invention provides a water-soluble composition including: (a) a solubilizing agent of the invention, and b) a organoleptic additive, selected from a fragrance, a flavoring agent and combinations thereof, wherein the composition has an organoleptic property enhanced relative to an essentially identical composition wherein said solubilizing agent is not present or is present in a concentration less than the concentration of the solubilizing agent in said water-soluble composition.

In a preferred embodiment, the solubilizing agent is water soluble. In one example, the solubilizing agent and the organoleptic additive form micelles when added to an aqueous solution. The particle sizes of these micelles can be determined using art recognized methods, such as light scattering techniques. In an exemplary embodiment, the micelles formed between the additive and the solubilizing agent, have a median (average) particle size of less than about 200 and preferably less than about 100 nm. In another example, the micelles formed between the additive and the solubilizing agent, have a median particle size of less than about 90 nm, less than about 80 nm, less than about 70 nm or less than about 60 nm. In a further example, the micelles formed between the additive and the solubilizing agent, have a median particle size of less than about 50 nm, less than about 40 nm or less than about 30 nm. In another exemplary embodiment, the average particle size is from about 10 nm to about 90 nm. Another exemplary average particle size is from about 5 nm to about 70 nm, preferably from about 10 nm to about 50 nm, more preferably from about 10 nm to about 30 nm. In a particular example, the micelles formed between the additive and the solubilizing agent, have a median particle size between about 30 nm and about 20 nm (e.g., about 25 nm). Smaller particle sizes are generally preferred. Preferred particle sizes are those that demonstrably enhance the organoleptic properties of the additive and, preferably the composition containing the organoleptic additive.

The term “water-soluble” refers to moieties that have a detectable degree of solubility in water. Methods to detect and/or quantify water solubility are well known in the art.

As used herein, “acceptable carrier” includes any material, which when combined with the additive and the solubilizing agent, does not have deleterious effects on the additive or solubilizing agent, and is preferably non-reactive with the immune system of the subject two whom the composition is administered (e.g., hypoallergenic). Examples include, but are not limited to, any of the standard carriers for flavors, fragrances and colors, e.g., a buffered phosphate solution, a buffered saline solution, water, emulsions such as oil/water emulsion, and various types of wetting agents. Other carriers may also include sterile solutions, tablets, e.g., coated tablets, and capsules (e.g., micro- nano-capsules). Such carriers optionally contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients that are not solubilized by the solubilizing agent. Compositions comprising such carriers are formulated by well known conventional methods.

The water-soluble compositions of the present invention contain an organoleptic additive and a solubilizing agent in an amount above the critical micelle concentration (e.g., about CMC; 0.2-0.3 mg/mL of solvent). In an exemplary formulation, a molar ratio of approximately 0.01:1 to 1:5 organoleptic to solubilizing agent is used. The upper limit of the molar ratio is not critical, and the solubilizing agent can be used in any excess.

The compositions of the present invention can be prepared by many different procedures, either in the presence or in the absence of an auxiliary organic solvent. In the first case, an organoleptic compound and a solubilizing agent are first dissolved in a predetermined molar ratio in a water-miscible organic solvent and this solution is then diluted with a predetermined amount of water, without precipitation of the organoleptic compound. The organic solvent and water are then removed by evaporation under reduced pressure. A volatile organic solvent is usually removed first, followed by water, in which case the amount of water removed from the solution may be controlled, to achieve a desired concentration of the composition in the remaining concentrate. Alternatively, both the organic solvent and water are removed by evaporation, and the waxy residue is reconstituted with a suitable aqueous medium (such as water, physiological saline, or a buffer solution), to provide a clear aqueous solution.

The organic solvent used is in the above procedure should be a good solvent for both the organoleptic compound and the solubilizing agent and is preferably miscible with water. If a composition is to be used in a pharmaceutical formulation, this solvent should be also pharmaceutically acceptable, as the removal of the solvent by evaporation may not always be possible. Examples of solvents suitable for the practice of the invention are tetrahydrofuran, ethanol, methanol, ethylene glycol, propylene glycol, and acetic acid. Solvents with a low boiling point, such as tetrahydrofuran, are preferred.

The amount of the organic solvent is not critical, and is equal to or greater than the minimum amount of solvent necessary to dissolve the given amounts of the organoleptic compound and solubilizing agent. The amount of water used for the dilution is also not critical, and is preferably between 10 to 25 times the volume of the organic solvent.

An alternative procedure for preparing compositions according to the invention consists of preparing first a mixture of a organoleptic compound and a solubilizing agent in a predetermined molar ratio. In one example, the mixture is heated. In another example, the mixture is heated to a temperature sufficient to produce a melt (e.g., higher than the respective melting points of the compound and the solubilizing agent), for a time necessary to obtain a clear melt, which process can be seen as a dissolution of the organoleptic compound in the solubilizing agent. The melt so obtained can be reconstituted with a predetermined amount of a suitable water-based carrier, to provide a clear aqueous solution of a desired concentration. This method of preparing compositions of the invention is preferred. In one example, the solubilizing agent is present in the resulting composition in an amount of at least about 0.001% by weight. Preferably the amount is about 0.01% to about 10% by weight, more preferably at least about 0.1%, 0.5%, or 1% (w/w). However, levels of up to about 20% by weight may be used in particular cases, depending on the additive. In one example, the ration of solubilizing agent (e.g., PTS) to organoleptic additive is between about 0.1:1 and about 10:1, preferably about 0.3:1 to about 5:1, and more preferably from about 0.3:1 to about 3:1.

The ability of solubilizing agents of the present invention to dissolve organoleptic compounds in the absence of an auxiliary organic solvent can be used for preparing water-soluble forms of organoleptic compounds.

Exemplary compositions of the present invention show an excellent solubility in water and allow the preparation of aqueous solutions of a wide range of concentrations. As the concentrated solutions can be diluted with an aqueous medium in any proportion and over a wide range of pH conditions without precipitation of the lipophilic compound, the solubility of the compound is maintained under physiological conditions, for example after an oral or parenteral administration of the composition. This normally results in an improved bioavailability of the compound.

Exemplary compositions of the present invention and aqueous solutions thereof show an excellent stability over long periods of time (several months at room temperature, at least one year when refrigerated, or indefinitely when frozen) and over wide ranges of temperature and pH conditions (temperatures from −80° C. to 120° C., pH from 2.0 to 8.0). Aqueous solutions can be repeatedly frozen and thawed without any perceptible degradation. Stability under high temperature conditions allows an easy sterilization of the solutions, without compromising the solubility of the active ingredient.

The compositions of the present invention can be incorporated into numerous formulations, including, but not limited to, beverages, foods, scented products, pharmaceutical or cosmetic formulations, which are then characterized by improved organoleptic properties of the active ingredient. A presently preferred formulation is a water-based formulation.

Exemplary formulations may further contain additional active ingredients and/or a pharmaceutically or cosmetically acceptable additives or vehicles, including solvents, adjuvants, excipients, sweeteners, fillers, colorants, flavoring agents, lubricants, binders, moisturizing agents, preservatives and mixtures thereof. The formulations may have a form suitable for a topical (e.g., a cream, lotion, gel, ointment, dermal adhesive patch), oral (e.g., a capsule, tablet, caplet, granulate), or parenteral (e.g., suppository, sterile solution) administration.

In another example, the invention provides a composition formed by a method comprising: (a) combining an organoleptic additive (e.g., a fragrance, a flavoring agent and combinations thereof) with a solubilizing agent of the invention (e.g., PTS), thereby forming an additive-solubilizing agent mixture; and (b) contacting the additive-solubilizing agent mixture with a water-based carrier (e.g., water).

### Methods

In a first aspect, the present invention provides a method of (detectably) enhancing an organoleptic property of a composition (e.g., an aqueous composition) that includes an organoleptic additive. In one example, the organoleptic property is a member selected from flavor, fragrance and combinations thereof. The method includes solubilizing the additive in a water-based carrier using a solubilizing agent, e.g., a solubilizing agent according to Formulae (I) to (VII). In one example, the solubilizing agent, the organoleptic additive and the water-based carrier form an emulsion comprising micelles formed between the solubilizing agent and the organoleptic additive.

In one example, the above emulsion is formed by a method that includes (a) combining the additive and the solubilizing agent, thereby forming an additive-solubilizing agent mixture; and (b) contacting the additive-solubilizing agent mixture with a water-based carrier.

The invention also provides a method of increasing the vapor pressure of an organoleptic additive in a water-based carrier (e.g., hydrophilic solvent, such as water). The method includes: contacting an organoleptic additive of the invention and a solubilizing agent of the invention with a water-based carrier, thereby solubilizing the organoleptic additive in the water-based carrier. Increased vapor pressure is measured relative to an essentially identical composition wherein the solubilizing agent is not present.

The invention also provides a method including: contacting a mixture of an organoleptic additive and a solubilizing agent of the invention with a water-based carrier forming a composition, wherein the organoleptic additive has a decreased vapor pressure, relative to an essentially identical composition wherein the solubilizing agent is not present.

This invention also provides a means for preserving the organoleptic properties of an organoleptic additive in a water-based composition. For example, composition life is prolonged when organoleptic additive release is delayed in a water-based composition. The method includes, mixing an organoleptic additive, a solubilizing agent, and a water-based carrier, thereby solubilizing the organoleptic additive in the water-based carrier. In one example, solubilization of the organoleptic additive preserves the organoleptic properties of the additive, and preferably preserves the enhanced organoleptic properties of the additive.

The invention also provides a method of making a water-based composition having improved organoleptic properties. The method includes, contacting an organoleptic additive of the invention and a solubilizing agent of the invention with a water-based carrier, thereby solubilizing the organoleptic additive in the water-based carrier. Solubilization of the organoleptic additive enhances the organoleptic properties of the additive. The invention also provides a water-based composition made by any of the above described methods.

In one example, according to any of the above embodiments, the organoleptic additive is first contacted with the solubilizing agent, optionally at elevated temperature (e.g., about 40° C., about 50° C., about 60° C., about 70° C., about 80° C., about 90° C., about 100° C. or greater than 100° C.) forming a mixture. The mixture is then contacted with the water-based carrier to dissolve the additive in the water-based carrier.

Exemplary organoleptic additives useful in any of the above embodiments are flavoring agents, fragrance additives (e.g., lipophilic flavoring agents or fragrance additives) and combinations thereof. Exemplary flavors and fragrances are disclosed herein. See, e.g., FIGS. 1 and 2. Exemplary solubilizing agents useful in the methods of the invention are also disclosed herein.

In one example, according to any of the above embodiments (methods and compositions) the additive has a concentration of at least 0.01%, at least 0.03%, at least 0.05%, at least 0.1%, at least 0.2%, at least 0.3%, at least 0.4%, at least 0.5%, at least 1%, at least 2%, at least 3%, at least 4% or at least 5% (w/w) in the emulsion, the water-based carrier or the water-based composition.

In one example, according to any of the above embodiments (methods and compositions), the emulsion, the water-based carrier or the water-based composition does not include a ubiquinone or a ubiquinol, which is not bound to at least one hydrophilic moiety Y¹ or Y². For example, the composition in any of the methods described herein does not include a compound with a structure according to the formula:

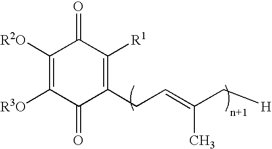

wherein R¹, R² and R³ are members independently selected from substituted or unsubstituted C₁-C₆ alkyl groups; and n is an integer from 0 to 19.

In one example according to any of the above embodiments, the method can further include removing water from the emulsion, water-based carrier or composition. In an exemplary embodiment, the water is removed (dried) to a solid form using methods known in the art. Such methods can include without limitation spray drying, nozzle drying (e.g., tower or fountain), wheel drying, flash drying, rotary wheel drying, oven/fluid bed drying, vacuum evaporation, freeze drying, drum drying, tray drying, belt drying, sonic drying, and the like. In one example, the method further includes spray-drying the emulsion, water-based carrier or composition, optionally in the presence of a water-soluble or water-insoluble additive. Exemplary additives include additional solubilizing agents of the invention or other solubilizing agents known in the art. In one example, the additive is cyclodextrin.

### Measurement of Organoleptic Properties

“Enhancing” or “enhancement” of an organoleptic property, e.g., flavor or fragrance, as used herein, refers to an intensifying of the sensory perception of the flavor or fragrance relative to the sensory perception of essentially the same amount of additive in an essentially identical composition that does not include the solubilizing agent (e.g., the additive solubilized by a solubilizing agent). In one example, enhancement of an organoleptic property is due to an increased vapor pressure of, e.g., a fragrant compound. In another example, enhancement of an organoleptic property is due to the small particle size of the micelles formed between the additive and the solubilizing agent when contacted with an aqueous carrier. For example, the fine dispersion of the additive can facilitate the interaction of the additive with, e.g., a taste receptor in the mouth of a human. “Enhancing” an organoleptic property can also mean “prolonging” or “preserving” a given property. For example, certain preparations loose their flavor or fragrance over time (e.g., due to vaporization). In one example, the “encapsulation” of the flavoring agent or fragrance due to the described formation of micelles can slow down the process of vaporization. In another example, the “encapsulation” into micelles can prevent or diminish the chemical modification (e.g., chemical degradation due to oxidative reactions) of flavor and fragrance components. In this respect, the invention also provides methods of preserving freshness of a flavoring agent or fragrance (e.g., in a product, such as a food or beverage).

Standard methods are available for testing the sensory perception of flavor and fragrance. Known in the art as flavor testing and sensory analysis, such analyses include flavor constituent testing, flavor profiling through sensory analysis and consumer surveys involving sensory analysis. Exemplary criteria for sensory analysis are provided at http://www.nysaes.cornell.edu/fst/faculty/acree/fs430/lectures/htl13sensoryprimer.html. See, also, The Role of Sensory Analysis in Quality Control, edited by June E. Yantis, is part of the ASTM Manual Series MNL14, and provides a basic guide to in-plant sensory testing; and another industry standard, Sensory Testing Methods, 2d Ed., ASTM Manual Series MNL26; Sensory Evaluation in Quality Control, by Alejandra M. Muñoz, Gail Vance Civille and B. Thomas Carr.

The present invention provides a method of producing a composition that is distinguishable by art-recognized methodologies and standards as having organoleptic properties enhanced relative to an essentially identical composition in the absence (or in the presence of a lesser amount) of the solubilizing agent.

Conventionally, the discrimination and evaluation of odors is performed by the olfactory sense of human beings. By this method, it must be considered that different persons (or panels) have different olfactory sensitivities and the olfactory sense of a panel may change depending on the physical condition on the day of the test. Therefore, to obtain an objective result with high accuracy, it is necessary to gather an adequate number of panels and to conduct the test under an adequately uniform environmental condition.

Some conventional methods use a gas chromatograph (GC) or a gas chromatograph/mass spectrometer (GC/MS) to analyze and discriminate the components of the odor concerned. These methods treat the odor as a volatile chemical substance and can effectively identify the causative agent of the odor. However, the conventional methods cannot correlate the odor composition with the organoleptic evaluation by the olfactory sense of the human being.

A conventional device of use in determining the enhancement of an organoleptic property of a composition of the invention, is a product of Gerstel (http://www.gerstelus.com), called the “Olfactory Detector Port (ODP-2).” ODP-2, which is an attachment for a gas chromatograph, allows a panel to smell the effluent sample separated by the column of the gas chromatograph and enter information about the odor intensity in real-time while the sample is being analyzed with the detector. The information entered by the panel is used to create a graph showing the change of the odor intensity with time. The relation between the chromatogram created by the gas chromatograph and the aforementioned graph enables the analysis on the relation between the odor composition and the organoleptic evaluation.

Another odor discriminating apparatus of use in confirming enhancement of organoleptic properties of compositions of the invention is disclosed in the Japanese Unexamined Patent Publication No. 2003-315298 and on the following website: http://www.an.shimadzu.co.jp/products/food/ffl.htm. The apparatus includes plural pieces of odor sensors having different response characteristics and calculates the quality and intensity of an odor by processing the detection signals of the odor sensors by a cluster analysis, a principal component analysis or other types of multivariate analysis, or by a non-linear analysis using neural networks. This type of odor discriminating apparatus treats an odor as a mixed odor and does not separate it into components, enabling the comparison and determination of mixed odors or the calculation of an odor index or other index indicating the odor intensity in terms of the olfactory sense of the human being.

### Solubilizing Agent

“Solubilizing agent”, as used herein, refers to a class of water soluble organic molecules that significantly increase the aqueous solubility of lipophilic compounds. Hydrotropes are similar to surfactants but typically possess a smaller hydrophobic moiety. An example of a hydrotrope is sodium xylenesulfonate, which is used in the consumer product industry. Other examples include nicotinamide, sodium ascorbate, cyclodextrins, liposomes and nanoparticles. Exemplary hydrotropes of the present invention include those disclosed in U.S. Pat. No. 6,632,443, and U.S. patent application Ser. No. 11/675,539, filed Feb. 15, 2007, WO2006/010370. An exemplary solubilizing agent is a conjugate formed between a glyceride (e.g., a mono-, di-, or tri-glyceride) and a Polysorbate, e.g., Polysorbate 80 (“Tween 80”).

An exemplary solubilizing agent of use in the compositions and methods of the invention has a structure according to Formula (I):

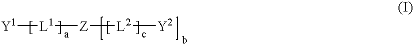

In Formula (I), a, b and c are integers independently selected from 0 and 1. In one example, b is 0. Z is a hydrophobic (lipophilic) moiety. In one example, Z is a sterol (e.g., beta-sitosterol, cholesterol, 7-dehydrocholesterol, campesterol, ergosterol, stigmasterol). In another example, Z is a tocopherol (e.g., alpha-tocopherols, β-, γ-, and δ-tocopherols, alpha-tocotrienol) or a derivative or homologue thereof. In yet another example, Z is a ubiquinol. A person of ordinary skill in the art will understand that the residue of the hydrophobic moiety is the entire hydrophobic molecule, except for at least one hydrogen atom, which is replaced with the hydrophilic moiety or the linker-hydrophilic moiety cassette (e.g., hydrogen atom of esterified hydroxyl group, such as 3-β-hydroxyl group of cholesterol or sitosterol or 6-hydroxyl group of α-tocopherol). Preferably, when b is 0 and Z is alpha-(+)-tocopherol, L¹ is not derived from succinic acid.

In Formula (I), Y¹ and Y² are linear or branched hydrophilic moieties comprising at least one polymeric moiety, wherein each polymeric moiety is independently selected. In one example, Y¹ and Y² are independently selected from hydrophilic (i.e., water-soluble) polymers. In another example, Y¹ and Y² are members independently selected from poly(alkylene oxides) (i.e., polyethers), polyanions, polycations, polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include poly(alkylene glycols), such as polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

The term “polyalkylene glycol” includes polymers of lower alkylene oxides, in particular polymers of ethylene oxide (polyethylene glycols) and propylene oxide (polypropylene glycols), having an esterifiable hydroxy group at least at one end of the polymer molecule, as well as derivatives of such polymers having esterifiable carboxy groups. The residue of the hydrophilic moiety is the entire hydrophilic molecule, except for the atom involved in forming the bond to the ubiquinol moiety or the linker moiety (i.e. an esterified hydroxy group, the oxygen molecule of an ether bond, a carboxy or amino group) or groups, such as terminal hydroxy groups of a polyethylene glycol molecule.

Polyethylene glycols are most particularly preferred for the practice of the present invention. Suitable polyethylene glycols may have a free hydroxy group at each end of the polymer molecule, or may have one hydroxy group etherified with a lower alkyl, e.g., a methyl group. Also suitable for the practice of the invention are derivatives of polyethylene glycols having esterifiable carboxy groups or amino groups, which may be used to form an amide bond. Polyethylene glycols are commercially available under the trade name PEG.

PEG is usually a mixture of oligomers characterized by an average molecular weight. In one example, the PEG has an average molecular weight from about 200 to about 5000. In another examplary embodiment, PEG has an average molecular weight from about 400 to about 4000. In another examplary embodiment, PEG has an average molecular weight from about 400 to about 2000. In another examplary embodiment, PEG has an average molecular weight from about 400 to about 1200. In another examplary embodiment, PEG has an average molecular weight from about 400 to about 1000. In one example, the lipophilic moiety of the solubilizing agent is PEG-400. In one example, the lipophilic moiety of the solubilizing agent is PEG-600. Both linear and branched PEG moieties can be used as the hydrophilic moiety of the solubilizing agent in the practice of the invention. In an exemplary embodiment, PEG has between 1000 and 5000 subunits. In an exemplary embodiment, PEG has between 100 and 500 subunits. In an exemplary embodiment, PEG has between 10 and 50 subunits. In an exemplary embodiment, PEG has between 1 and 25 subunits. In an exemplary embodiment, PEG has between 15 and 25 subunits. In an exemplary embodiment, PEG has between 5 and 100 subunits. In an exemplary embodiment, PEG has between 1 and 500 subunits.

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660 and WO 02/09766; as well as Kodera Y., Bioconjugate Chemistry 5: 283-288 (1994); and Yamasaki et al., Agric. Biol. Chem., 52: 2125-2127, 1998, all of which are incorporated herein by reference in their entirety. Exemplary branched PEG moieties involve a branched core molecule having at least two PEG arms attached, each at a different attachment point.

The hydrophilic moiety used to make the solubilizing agent is a hydrophilic molecule having a functional group, which can be used to chemically attach the hydrophilic molecule to the hydrophobic moiety, either directly or through a linker moiety. Examples of said functional group include esterifiable hydroxy groups, carboxy groups, and amino groups.

In Formula (I), L¹ and L² are linker moieties. In one example, L¹ and L² are independently selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl.

In one example, at least one of L¹ and L² includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups.

In another example according to any of the above embodiments, at least one of L¹ and L² includes a moiety having the following formula:

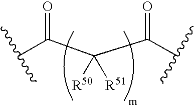

wherein m is an integer selected from 1 to 30. In one example, m is selected from 2 to 20. In another embodiment, m is not 2. Each R⁵⁰ and each R⁵¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In one example, R⁵⁰ and R⁵¹ are both H. The linker can be derived from an alkanedioic acid of the general formula HOOC—(CH₂)ₘ—COOH. In one example, m is not 2. Preferred linkers include diesters derived from an alkanedioic acid. For the practice of the present invention, alkanedioic acids with m from 0 to 18 are preferred, those with m from 6 to 10 being particularly preferred. In some embodiments, sebacic acid (m=8) is particularly preferred.

Other preferred linkers include diethers derived from a substituted alkane. In an exemplary embodiment the substituted alkane has the general structure X—(CH₂)ₙ—X′ wherein X and X′ independently represent a leaving group such as a halogen atom or a tosylate group. For the practice of the present invention, substituted alkanes with n from 0 to 18 are preferred, those with n from 6 to 10 being particularly preferred. The ether derived from a 1,10-substituted decane (n=10), such as 1,10-dibromodecane is most particularly preferred.

Other exemplary solubilizing agents, such as polyoxyethanyl-tocopheryl-sebacate (PTS), polyoxyethanyl-sitosterol-sebacate (PSS), polyoxyethanyl-cholesterol-sebacate (PCS) are disclosed in U.S. Pat. No. 6,191,172, U.S. Pat. No. 6,632,443 and WO96/17626 the disclosures of which are incorporated herein by reference for all purposes.

The compounds of Formula (I) can be prepared by standard methods of synthetic organic chemistry, well known to those skilled in the art. In particular, compounds where p is equal to 1 or 2 and m is equal to 1 can be prepared by reacting a compound of the general formula Z-OH with a compound of the general formula X—OC—(CH₂)ₙ—CO—X, where X is a leaving group, and further reacting the product so obtained with a compound of the general formula HO—Y—OR, wherein R is hydrogen or an alkyl, and Z, Y and n are as defined hereinbefore. Halogens, in particular Cl and Br, are preferred as the leaving group X. Hydrogen and lower alkyl (C₁-C₄) are preferred for R.

In one exemplary embodiment, the solubilizing agent has a structure according to one of the following formulae:

Y¹-Z-Y²;

Y¹-L¹-Z-Y²;

Y¹-Z-L²-Y²; and

Y¹-L¹-Z-L²-Y²

wherein a, Y¹, Z and L¹ are defined as herein above. All embodiments described herein above for Formula (I) equally apply to the examples of this paragraph.

In another example according to any of the above embodiments, the solubilizing agent has a structure according to Formula (II), wherein the integer a, Y¹, Z and L¹ are defined as herein above:

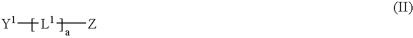

All embodiments described herein above for Formula (I) equally apply to compositions of Formula (II).

### Solubilizing Agents Wherein Z is a Sterol

In an exemplary embodiment, in Formula (I) or (II), Z has a structure according to the following formula:

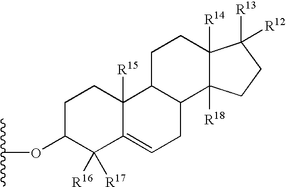

wherein R¹² and R¹³ are selected from H and substituted or unsubstituted alkyl, wherein at least one of R¹² and R¹³ is substituted or unsubstituted alkyl. R¹⁴, R¹⁵, R¹⁶, R¹⁷, and R¹⁸ are independently H, or substituted or unsubstituted alkyl. In one example, the sterol is selected from 7-dehydrocholesterol, campesterol, sitosterol, ergosterol and stigmasterol. Cholesterol and sitosterol are preferred sterols, sitosterol being particularly preferred. In an exemplary embodiment, Z is member selected from a zoosterol and a phytosterol.

### Solubilizing Agents Wherein Z is a Tocopherol or a Tocotrienol

In one example, Z in Formula (I) or (II) has a structure according to the following formulae:

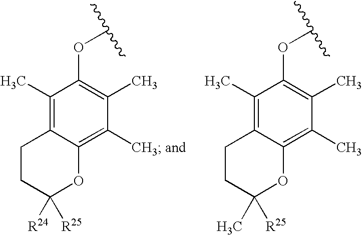

wherein R²⁰, R²¹, R²², R²³, R²⁴ and R²⁵ are members selected from H, halogen, nitro, cyano, OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁶, R¹⁷, and R¹⁸ are independently H, or substituted or unsubstituted alkyl. In an exemplary embodiment, at least one of R²⁴ and R²⁵ comprises an isoprene moiety. In another example, R²⁵ is a member selected from substituted or unsubstituted alkyl and substituted or unsubstituted heteroalkyl. In one example, R²⁴ is methyl. In another example, R²⁵ includes a moiety having a structure selected from the following formulae:

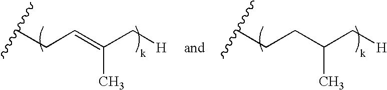

wherein k is an integer selected from 1 to 12. In an exemplary embodiment, k is from 2 to 6. In another exemplary embodiment, k is 3.

### Solubilizing Agents Wherein Z is Ubiquinol

In an exemplary embodiment, Z in Formula (I) or (II) is an ubiquinol. In another exemplary embodiment one or both of the phenolic hydroxy groups of the ubiquinol are derivatized with a hydrophilic moiety of the invention. In an exemplary embodiment, the solubilizing agent has a structure according to the Formula (III):

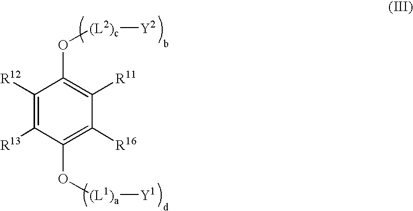

In Formula (III), L¹, L², Y¹ and Y² are defined as herein above. R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹² and R¹³, along with the atoms to which they are attached, are optionally joined to form a 4- to 8-membered ring.

In one example, in Formula (III), L¹ and L² are linker moieties, which are members independently selected from substituted or unsubstituted alkyl and substituted or unsubstituted heteroalkyl. In another example, Y¹ and Y² are polymeric hydrophilic moieties, which are members independently selected from polyethers, polyalcohols and derivatives thereof. In one embodiment, Y¹, Y², L¹ and L² do not comprise a labeling moiety, a targeting moiety or a drug moiety. In Formula (III), the indices a, b, c and d are members independently selected from 0 and 1 with the proviso that at least one of b and d is 1. When b is 0, ((L²)_{c}-Y²)_{b} is preferably a member selected from H, a negative charge, and a salt counterion. When d is 0, ((L¹)ₐ-Y¹)_{d} is preferably a member selected from H, a negative charge, and a salt counterion.

In an exemplary embodiment, in Formula (III), R¹⁶ includes a moiety having a structure selected from the following formulae:

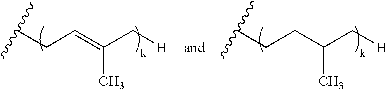

wherein k is an integer selected from 1 to 20. In an exemplary embodiment, k is an integer selected from 6, 7, 8, 9, 10, 11 and 12. In another exemplary embodiment, k is 10.

In an exemplary embodiment, in Formula (III), R¹¹, R¹² and R¹³ are members independently selected from H, unsubstituted alkyl (e.g., methyl, ethyl), alkoxy (e.g., methoxy, t-butoxy), halogen substituted alkoxy and halogen-substituted alkyl (e.g., CF₃). In one example, R¹¹ is H. In another embodiment of the invention, in Formula (III), R¹¹ is a methyl group. In a particular example, R¹¹ is methyl and R¹² and R¹³ are both methoxy.

An exemplary solubilizing agent according to Formula (III) has a structure according to Formula (IV):

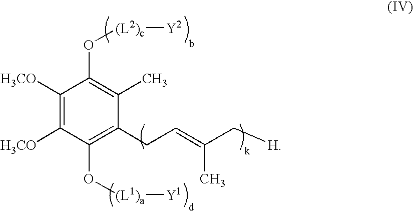

In another example according to any of the above embodiments, one of the phenolic hydroxy groups of the ubiquinol analog is derivatized with a hydrophilic moiety of the invention. Exemplary solubilizing agents have the structure:

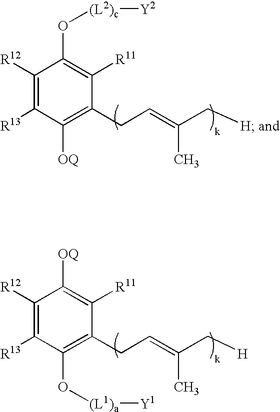

wherein Q is a member selected from H, a negative charge and a salt counter ion.

Exemplary solubilizing agents have a structure according to Formula (V), Formula (VI) or Formula (VII):

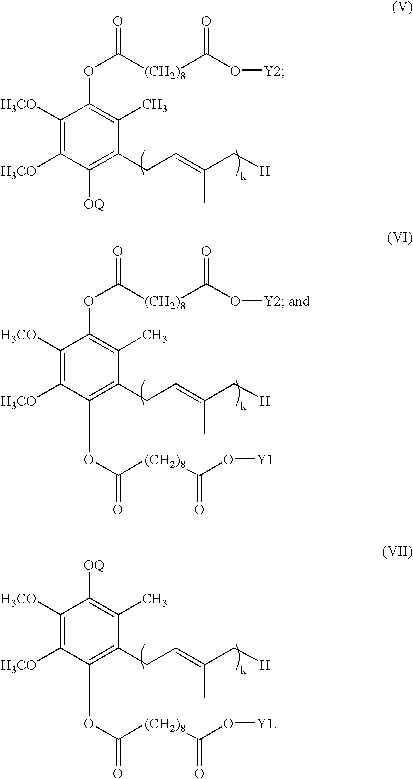

Other exemplary components of use in the methods and compositions of the present invention are disclosed in commonly-owned, copending U.S. patent application Ser. No. 12/024,936, U.S. Provisional Patent Application No. 60/887,754, filed on Feb. 1, 2007 and U.S. Provisional Patent Application No. 60/947,943, filed on Jul. 3, 2007, the disclosures of which are incorporated herein by reference in their entirety for all purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### EXAMPLES

### Example 1

### Solubilization of Citrus and Orange Oils

Citrus oil and orange oil were stably solubilized in an aqueous carrier (e.g., water) at the indicated ratios using PTS. PTS and the respective flavor oil were mixed in a 15 or 50 ml plastic tube at ˜40° C. Some water was added and the samples were vortexed to produce a dispersion. Additional water in an amount sufficient to produce the indicated concentration of the PTS/flavor mix was then added. The samples were vigorously shaken on a mechanical shaker for about 10 hours at ambient temperature (e.g., 20-25° C.). Exemplary composition thus produced are summarized in Table 1, below.

TABLE 1
Exemplary Citrus- and Orange Oil Compositions

| | | Ratio | Concentration | Volume |
|---|---|---|---|---|
| No. | | PTS:Flavor Oil [w/w] | [mg/mL] | [mL] |
| 1 | Citrus | 0.3:1/0.5:1/1:1/2:1/3:1 | 10.0 | 10 |
| 2 | Citrus | 0.3:1/0.5:1/1:1/2:1/3:1 | 3.0 | 10 |
| 3 | Citrus | 0.3:1/0.5:1/1:1/2:1/3:1 | 1.0 | 10/50 |
| 4 | Orange | 0.3:1/0.5:1/1:1/2:1/3:1 | 1.0 | 50 |

Results: All 10 mg/ml conc. were found to be opaque. All 1 mg/ml samples (0.1% w/w) at 0.3:1 were clear. At higher PTS concentration (e.g., 1:1, 2:1 and 3:1), the samples were increasingly opaque. In general, all samples after preparation are opaque or slightly opaque and clarified when kept at ambient temperature for about 10 hours. Shaking (mechanical shaker) sped the clearing process (e.g., 1 or 2 h at ambient temperature). Thus, 0.3:1 or 0.5:1 formulations are preferred over higher PTS ratios. 1 mg/ml and 3 mg/ml solutions became clear faster and are stable.

### Example 2

### Solubilization of Strawberry and Cranberry Fragrance

Components were mixed in Eppendorf tubes at ˜40° C., vortexed for 30 sec, spinned down, the appropriate amounts of the mixtures were than transferred to the larger tubes and water was slowly added when vortexed. Strawberry formulations were slightly opaque but clarified when incubated at about 4° C. to ambient temperature for about 10 hours. Alternatively, opaque solutions were agitated on a mechanical shaker for 5 h at ambient temperature for clarification. All cranberry formulations were opaque to slightly opaque and did not clarify entirely when incubated at ambient temperature or when being refrigerated. Exemplary compositions are summarized in Table 2, below.

TABLE 2
Exemplary Citrus- and Orange Oil Compositions

| | | Ratio | Concentration | Volume |
|---|---|---|---|---|
| No. | | PTS:Fragrance [w/w] | [mg/mL] | [mL] |
| 1 | Strawberry | 0.3:1 | 1/2/5 | 40 |
| 2 | Cranberry | 0.3:1/0.5:1/1:1/2:1 | 1 | 40 |

### Example 3

### Solubilization of Menthol

### Procedure 1:

Equal amounts of menthol (e.g., 52.25 g) and PTS (e.g., 52.25 g) were mixed at 60° C. To the samples was added water (e.g., 45 g) and the samples were agitated for 5 h resulting in an opaque-opalescent solution. After 5 h an aliquot was removed and diluted 5 times with water to result in 10 mg/ml samples. Both stock and diluted samples were unstable due to slow crystallization of menthol during storage both at ambient temperature or when refrigerated.

### Procedure 2:

2.a) Menthol (2.0 g), PTS (2.0 g) and Miglycol (2.0 g) were mixed at 60-75° C. Water (34.0 g) was added. Samples were easily dispersible during 2-3 min at various temperatures (e.g., 80° C.-0° C.). This formulation (at 50 mg/ml) was opaque but stable for at least 4 month when refrigerated (about 4° C.).

2.b) Menthol (2.0 g), PTS (3.0 g), Miglycol (2.0 g) were mixed at 60-75° C. Water (33.0 g) was added. Samples were easily dispersible during 2-3 min at various temperatures (80° C.-0° C.). This formulation (at 50 mg/ml) was slightly opaque, but stable for at least 4 month when refrigerated.

2.c) Menthol (2.0 g), PTS (4.0 g), Miglycol (2.0 g) were mixed at 60-75° C. Water (32.0 g) was added. Samples were easily dispersible during 2-3 min at various temperatures (80° C.-0° C.). This formulation (at 50 mg/ml) was clear and stable for at least 4 month when refrigerated.

What is claimed is:

1. A method of enhancing an organoleptic property of a composition comprising an organoleptic additive, which is a member selected from a fragrance, a flavoring agent and combinations thereof, wherein said organoleptic property is a member selected from flavor, fragrance and combinations thereof, said method comprising:
forming an emulsion of said additive in a water-based carrier using a solubilizing agent having a structure according to Formula (I): 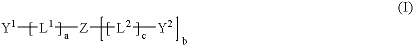
wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl,
thereby enhancing said flavor, fragrance or a combination thereof, of said composition.

2. The method of claim 1, further comprising contacting said emulsion with a member selected from a food, a chewing gum base, a beverage, a pharmaceutical composition, an oral hygiene product, a skin-care product and a detergent.

3. The method of claim 2, wherein said beverage is a member selected from carbonated or non-carbonated flavored waters, caffeinated or non-caffeinated soft drinks and juices.

4. The method of claim 1, further comprising removing water from said emulsion.

5. The method of claim 1, wherein said solubilizing agent has a structure according to Formula (II):

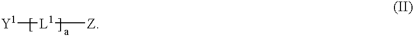

6. The method of claim 1, wherein said solubilizing agent is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

7. The method of claim 6, wherein said solubilizing agent is PTS.

8. The method of claim 1, wherein said water-based carrier is water.

9. The method of claim 1, wherein said additive is solubilized in said emulsion in the form of micelles formed between said additive and said solubilizing agent, wherein said micelles have a median particle size of less than about 60 nm.

10. The method of claim 1, wherein said emulsion includes at least 0.03% (w/w) of said additive.

11. The method of claim 1, wherein said solubilizing agent has a structure according to Formula (III): 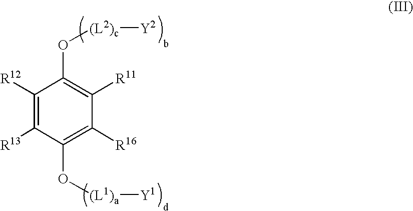
wherein
a, b, c and d are integers independently selected from 0 and 1, with the proviso that at least one of b and d is 1;
R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl,
wherein
R¹² and R¹³, together with the atoms to which they are attached, are optionally joined to form a 4- to 8-membered ring; and
R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl
wherein
R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

12. The method according to claim 1, wherein at least one of said Y¹ and Y² is a polyether.

13. The method according to claim 12, wherein said polyether is polyethylene glycol.

14. The method according to claim 13, wherein the polyethylene glycol has an average molecular weight of from about 200 to about 4000 Da.

15. The method according to claim 1, wherein said fragrance or flavoring agent is a member selected from geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenyl-ethanol, 2-phenylethyl acetate, benzyl alcohol, benzyl acetate, benzyl salicylate, styrallyl acetate, benzyl benzoate, amyl salicylate, dimethylbenzyl-carbinol, trichloromethylphenyl-carbinyl acetate, p-tert-butylcyclohexyl acetate, isononyl acetate, vetiveryl acetate, vetiverol, α-hexylcinnamaldehyde, 2-methyl-3-(p-tert-butylphenyl)propanal, 2-methyl-3-(p-isopropylphenyl)propanal, 2-(p-tert-butylphenyl)-propanal, 2,4-dimethyl-cyclohex-3-enyl-carboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyl-tetrahydropyran, 3-carboxymethyl-2-pentylcyclopentane, 2-n-heptylcyclopentanone, 3-methyl-2-pentyl-2-cyclopentenone, n-decanal, n-dodecanal, 9-decenol-1, phenoxyethyl isobutyrate, phenylacetaldehyde dimethylacetal, phenylacetaldehyde diethylacetal, geranyl nitrile, citronellyl nitrile, cedryl acetate, 3-isocamphylcyclohexanol, cedryl methyl ether, isolongifolanone, aubepine nitrile, aubepine, heliotropin, coumarin, eugenol, vanillin, diphenyl oxide, hydroxycitronellal, ionones, methylionones, isomethylionones, irones, cis-3-hexenol and esters thereof, indan musks, tetralin musks, isochroman musks, macrocyclic ketones, macrolactone musks, ethylene brassylate, ellagic acid, gallic acid and syringaldehyde.

16. The method of claim 1 comprising:
(a) combining said additive and said solubilizing agent, thereby forming an additive-solubilizing agent mixture; and
(b) contacting said additive-solubilizing agent mixture with a water-based carrier, thereby forming said emulsion.

17. A composition formed by a method comprising:
(a) combining an organoleptic additive, which is a member selected from a fragrance, a flavoring agent and combinations thereof, with a solubilizing agent, thereby forming an additive-solubilizing agent mixture, wherein said solubilizing agent has a structure according to Formula (I): 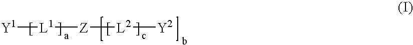
wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl; and
(b) contacting said additive-solubilizing agent mixture with a water-based carrier.

18. A water-soluble composition comprising:
a) a solubilizing agent having a structure according to Formula (I): 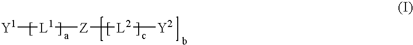
wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl; and
b) an organoleptic additive, selected from a fragrance, a flavoring agent and combinations thereof,
wherein said composition has an organoleptic property enhanced relative to an essentially identical composition wherein said solubilizing agent is not present or is present in a concentration less than the concentration of said solubilizing agent in said water-soluble composition.

19. The water-soluble composition according to claim 18, further comprising a water-soluble component selected from a solvent, an adjuvant, a sweetener, a filler, a colorant, a flavoring agent, a lubricant, a binder, a moisturizing agent, a preservative and mixtures thereof.

20. The water-soluble composition of claim 18 comprising at least 0.03% (w/w) of said additive.

21. A method comprising: contacting an organoleptic additive and a solubilizing agent having a structure according to Formula (I): 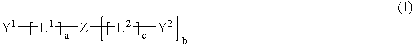
wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl,
with a water-based carrier forming a composition, wherein said organoleptic additive has a decreased vapor pressure, relative to an essentially identical composition wherein said solubilizing agent is not present.

22. A method of preserving an organoleptic property of an organoleptic additive in a water-based carrier, said method comprising:
(a) mixing said organoleptic additive and a solubilizing agent having a structure according to Formula (I): 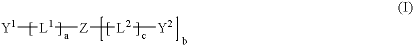
wherein
a, b and c are integers independently selected from 0 and 1;
Z is a member selected from a sterol, a tocopherol, a ubiquinol and derivatives or homologues thereof,
Y¹ and Y² are members independently selected from linear or branched hydrophilic moieties comprising at least one polymeric moiety, wherein each of said polymeric moiety is a member independently selected from poly(alkylene oxides) and polyalcohols; and
L¹ and L² are linker moieties independently selected from substituted or unsubstituted alkyl and substituted or unsubstituted heteroalkyl,
thereby forming a mixture; and
(b) contacting said mixture with said water-based carrier,
thereby preserving said organoleptic property.
